(19) United States
(12) Patent Application Publication
(10) Pub. No.: US 2019/0192725 A1
(43) Pub. Date: Jun. 27, 2019

(54) MAGNESIUM PHOSPHATE BIOMATERIALS

(71) Applicant: BONESTONE LTD., Longwell Green Bristol (GB)

(72) Inventors: Jake Edward Barralet, Montreal (CA); Faleh Ahmad Tamimi Marino, Montreal (CA); Andrew Paul Flynn, Mississauga (CA)

(21) Appl. No.: 15/963,053

(22) Filed: Apr. 25, 2018

Related U.S. Application Data
(63) Continuation of application No. 14/342,027, filed on Jun. 23, 2014, now abandoned.
(60) Provisional application No. 61/529,534, filed on Aug. 31, 2011.

(51) Int. Cl. A61L 24/02; A61L 27/12; C04B 28/30; A61L 24/00; C04B 9/04; C09D 1/06; C04B 12/02; C04B 28/34

(52) U.S. Cl. CPC A61L 24/02; A61L 27/12; C04B 28/30; A61L 24/0042; C04B 2111/00836; C09D 1/06; C04B 12/025; C04B 28/34; C04B 9/04

(57) ABSTRACT

There is provided a solid cement reactant comprising a dehydrated magnesium phosphate, and/or an amorphous or partially amorphous magnesium phosphate, and/or Farringtonite.

### CROSS REFERENCE TO RELATED APPLICATIONS

This application is a continuation of U.S. patent application Ser. No. 14/342,027, filed on Jun. 23, 2014, which is a National Entry Application of PCT Application No. PCT/CA2012/050606 filed on Aug. 31, 2012, and published in English under PCT Article 21 (2), which itself claims benefit of U.S. provisional application Ser. No. 61/529,534, filed on Aug. 31, 2011. All documents above are incorporated herein in their entirety by reference.

### FIELD OF THE INVENTION

The present invention relates to magnesium phosphate biomaterials, more particularly amorphous and partially amorphous magnesium phosphates, and cements comprising same as a reactant. The present invention is concerned with the use of this cement for bone repair and as a coating.

### BACKGROUND OF THE INVENTION

Bone is a dynamic system, required not only for support and movement, but also for the regulation of calcium and phosphate in the body. Bones also play a role in the production of blood cells via the bone marrow. Healthy bone is a self-restorative tissue, able to heal and adapt itself in the presence of fracture or changing load. It is when bone is not healthy or damage is too extensive that intervention is required to restore it to its optimal state. While many materials exist for the repair and augmentation of bone, some of which have been used for nearly five decades, they have mostly failed to meet their chief requirement; to restore bone to its natural state. While these materials may be able to provide support, repair, return aesthetics and augment bone, many suffer from one flaw: appropriate residency. For many of these materials, it is their lack of resorption within the body which is the problem, with many of them remaining long after the surrounding bone has healed. For others, it is their rapid resorption that causes loss of mechanical support or templating for the new growing bone.

Autologous bone grafts (autografts) are considered by many to be the gold standard in graft material. Harvested from the patient, this material is osteogenic, osteoconductive and osteoinductive; able to undergo complete resorption and remodeling at the implantation site. While these grafts are considered to be the best material for implantation site healing, bone integration and remodeling, they also suffer from nagging complications at the patient harvest site with complication rates reported at 8.5-20%.

Allogeneic graft materials (allografts) are a materials harvested from members of the same species. One third of bone grafts used in North America are allografts. The harvested material is osteoconductive and is believed to have some osteoinductivity due to residual growth factors remaining in the graft. While allografts have provided a solution to problems associated with the harvest of autograft material, they suffer from limitations of their own. Processing of allografts has come under fire for fear of disease transmission through implantation, while processing and sterilizing result in inconsistent osteoinductivity in a material that already suffers from limited resorption.

Xenogeneic bone grafts (xenograft) are derived from non-human species. The most common of these materials are bovine and coralline hydroxyapatite. Bovine material suffers from the same lack of resorption and potential for disease transmittance as found with allogeneic material. Coralline hydroxyapatite is created through a chemical reaction which converts the natural porous calcium carbonate structure of coral into hydroxyapatite preserving the cancellous bone-like architecture.

Synthetic materials for bone repair encompass a wide variety of material classes including metals, polymers and ceramics. First, metals comprise a large group of materials, which are often used for the stabilization and replacement of bone structures due to fracture, disease and wear. Stainless steel, commercially pure titanium, titanium alloys and cobalt alloys are all used in the manufacture of orthopaedic devices in the form of plates, screws, and joint replacement components. Though metallic biomaterials are able to provide excellent support their high strength is one of their weaknesses, with elastic modulae an order of magnitude greater than cortical bone, they do not allow the natural loading of the bone during healing. In the initial stages of fracture healing, this lack of loading is desired and allows the healing bone to regain its strength. However, in the later stages a condition known as “stress shielding” may develop. The lack of bone loading can lead to osteoporosis of the bone at the site of implantation. Additional issues with metallic biomaterials arise in the form of wear debris and corrosion products.

Polymers used in medicine are a mix of both natural and synthetic materials which have found applications in the form of cements, screws, plates, patches, lenses, tissue scaffolds, sutures, bearing surfaces and bandages. In orthopaedic applications, polymers have been used primarily for cementing of implants (PMMA) and bearing surfaces in joint replacement applications (UHMWPE). Resorbable polymers have been investigated for the replacement of metallic components to reduce stress shielding of healing bone. While tailoring of the polymers can optimize their in vivo degradation rates to that of healing bone, they lack the strength required to stabilize the bone as they degrade.

Ceramic materials have found a wide variety of applications in orthopaedics, specifically in situations requiring a stiff, high strength, wear resistant materials. Ceramics have traditionally been used as the bearing surfaces for joint replacements and in implant dentistry for tooth replacement. Ceramic materials are brittle solids, strong in compression and weak in tension; they are prone to catastrophic failure upon crack initiation. This inherent weakness in these materials has limited their applications to compressive or non-load bearing applications. Due to the natural presence of calcium in bone, calcium-based ceramics have been investigated for use in bone applications, principally calcium sulphates and calcium phosphates. These materials are prepared through a variety of methods and in a variety of forms, and have been shown to elicit low immune responses and have osteoconductive properties.

Cements give a surgeon the ability to form a material in situ allowing him/her to customize the material location, volume and shape.

While PMMA is not strictly a cement, as it does not set from a liquid and solid phase to form a ceramic, it is called bone cement and has been for decades due to its use in cementing orthopaedic devices. PMMA is a non-resorbable polymer and is only suitable for applications where resorption and bone regeneration are not required. During its polymerization however, the setting reaction consumes monomer in the setting liquid. This reaction is an exothermic event, generating temperatures of 40-50° C. in vivo, which can cause cell necrosis at the implantation site. In addition, the monomer in the liquid phase is not entirely consumed and can cause irreversible damage to the surrounding cells and reduce healing. After curing, fragments of the cement may be generated during normal wear and tear. These fragments stimulate the cells of the immune system which can stimulate an enzymatic release leading to bone resorption.

Calcium phosphates have received substantial attention as bone cements. The most widely used cements set to form hydroxyapatite (HA). It was one of the first materials to be investigated, as it is the natural mineral phase of bone. It proved to be a highly osteoconductive material, however due to its low solubility it did not resorb and remodel in vivo as hoped. Additional calcium phosphates have been investigated for their ability to fill in the performance gaps created by HA. Beta tricalcium phosphate is formed at high temperature and has a higher solubility than HA. Due to the required high temperature processing it must be processed into shapes prior to implantation and cannot be formed in situ. It can however be used as a reactant or filler in other cements.

Brushite (CaHPO₄.2H₂O) is an acidic calcium phosphate formed from a mixture of beta tricalcium phosphate (β-TCP) and monocalcium phosphate monohydrate (MCPM). It was found that brushite is inherently unstable within the body and over time undergoes a phase transformation into HA. In addition, the acidic nature of the cement leads to a low pH during the setting reaction which may lead to necrosis at the site of material implantation. Monetite (CaHPO₄) is a calcium phosphate mineral phase created through the autoclaving of brushite. Though this material cannot be mixed and set in situ as brushite can, it does not suffer from the phase transformation to HA in vivo and proceeds with a slow and controlled dissolution.

Calcium sulphate hemihydrate (CaSO₄.1/2H₂O), also known as Plaster of Paris, is likely the oldest inorganic cement used for the fixation, repair and augmentation of the bone. Due to the highly soluble nature of the material, its efficacy in orthopaedics has come into question.

On another subject, magnesium and its salts have not undergone a great deal of investigation as potential materials for bone augmentation due to previously poor results using magnesium metal and its alloys. Magnesium-based materials were first used in 1907, with the implantation of a magnesium plate to secure a fracture. The poor corrosion resistance of magnesium showed as the plate disintegrated after only 8 days and that its corrosion produced a large volume of by-product hydrogen gas beneath the skin. To improve the corrosion resistance, multiple materials have been investigated to alloy with magnesium. Though these materials showed slower corrosion times and maintained their mechanical strength, they still developed gas pockets, which must be drawn off by subcutaneous needle.

Most investigation into the use of magnesium and magnesium minerals has been directed toward integration into previous calcium phosphate systems. Previous studies have investigated calcium-magnesium phosphate cements with the addition of a struvite phase into hydroxyapatite resulting in a high strength material with greater bone volume development in vivo. Previous cements were made by mixing magnesium oxide with an acid or acid phosphates. Depending on the acid or acid phosphate used, the resultant products were newberyite (MgHPO₄.3H₂O), struvite (MgNH₄PO₄.6H₂O), schertelite (Mg(NH₄)₂(HPO₄)₂.4H₂O) or magnesium potassium phosphate hexahydrate (MgKPO₄.6H₂O)

The only FDA approved magnesium phosphate-based cement is OsteoCrete, created by Bone Solutions Inc. OsteoCrete is composed of magnesium oxide, monopotassium phosphate and a small amount of tricalcium phosphate which, when mixed with water, sets to form a magnesium potassium phosphate material.

Many factors influence the ability of a bone cement to be used effectively in a clinical setting. In addition to biocompatibility and osteoconductivity, the cement must possess adequate handling properties to allow surgeons to use it effectively and provide the patient with maximum benefit. Ideally, the cement must mix easily, have a setting time rapid enough to set shortly after implantation but allow the surgeon time to ensure proper placement, and be cohesive enough to remain at the site of implantation.

The liquid phase has many effects on the properties of cements. While many cements will readily mix and set with water, the use of various solutions can have a effects on the setting time, compressive strength and injectability. Many additives have been used to enhance both the setting reaction, strength and injectability of calcium phosphate cements.

The present description refers to a number of documents, the content of which is herein incorporated by reference in their entirety.

### SUMMARY OF THE INVENTION

More specifically, in accordance with the present invention, there is provided:
1. A solid cement reactant comprising:
   a dehydrated magnesium phosphate, and/or
   an amorphous or partially amorphous magnesium phosphate, and/or
   Farringtonite.
2. The solid cement reactant of claim 1 being a dehydrated magnesium phosphate that is also amorphous or partially amorphous.
3. The solid cement reactant of item 1 or 2, wherein the dehydrated magnesium phosphate is least 10% dehydrated.
4. The solid cement reactant of any one of items 1 to 3, wherein the dehydrated magnesium phosphate is completely dehydrated.
5. The solid cement reactant of item 1 or 2, wherein the amorphous or partially amorphous magnesium phosphate is obtained by heat-treatment of a magnesium phosphate.
6. The solid cement reactant of item 5, wherein the magnesium phosphate is magnesium phosphate pentahydrate.
7. The solid cement reactant of item 5 or 6, wherein the heat treatment is heating at a temperature between 400 and 800° C., preferably 600° C., for about 30 minutes.
8. The solid cement reactant of any one of items 1 to 7 further comprising an organic acid or a salt thereof.
9. The solid cement reactant of item 8, wherein the organic acid or salt thereof is citric acid or a citrate salt, such a sodium citrate.
10. The solid cement reactant of item 9 comprising citric acid in a concentration ranging between about 2 and about 20 wt % based on the total weight of the solid cement reactant.
11. The solid cement reactant of any one of items 1 to 10 comprising a soluble salt in a quantity that would be sufficient to produce an aqueous solution with a pH between about 3 and about 9 in an amount corresponding to the amount of a liquid cement reactant intended to the used with the solid cement reactant.
12. A cement mixture obtained by mixing the solid cement reactant of any one of items 1 to 11 with a liquid cement reactant.
13. A set cement obtained upon setting of the cement mixture of item 12.
14. The set cement or cement mixture of item 12 or 13, wherein the liquid cement reactant is an aqueous solution containing:
   organic acid ions, such as citrate ions, and/or
   monovalent cations, such as sodium ions, and/or
   phosphate ions.
15. The set cement or cement mixture of any one of items 12 to 14, wherein the liquid cement reactant is a buffer solution.
16. The set cement or cement mixture of any one of items 12 to 15, wherein the liquid cement reactant has a pH between about 1 and about 11, preferably between about 3 and about 10, more preferably between about 4 and about 9, and even more preferably between about 4 and about 8.
17. The set cement or cement mixture of any one of items 12 to 16, wherein the liquid cement reactant comprises a citrate solution, for example a sodium citrate solution.
18. The set cement or cement mixture of item 17, wherein the citrate solution has a pH between about 4 and about 8, preferably about 5.1.
19. The set cement or cement mixture of any one of items 12 to 18, wherein the liquid cement reactant comprises a phosphate solution, for example a sodium phosphate solution.
20. The set cement or cement mixture of item 19, wherein the phosphate solution has a pH is between about 4 and about 8, preferably 7.
21. The set cement of any one of items 13 to 20 having a crystalline phase that includes Farringtonite.
22. The set cement of any one of items 13 to 21 having a crystalline phase that includes Farringtonite and Newberyite.
23. The set cement of any one of items 13 to 22 displaying an exothermic peak between about 600 and about 700° C., when analyzed by thermal analysis.
24. The cement mixture of any one of item 12 and 14 to 20 comprising an amorphous magnesium phosphate, alkali metal ions and an aqueous solution.
25. A kit comprising the solid cement reactant of any one of items 1 to 11.
26. The kit of item 25 further comprising:
   a liquid cement reactant as defined in any one of item 12 and 14 to 20 or a component to be mixed with water or an aqueous liquid to form a liquid cement reactant as defined in any one of item 12 and 14 to 20, and/or
   instructions to effect a cement setting reaction.
27. The kit of item claim 25 or 26 further comprising one or more devices for mixing reactants and/or for delivery or application of a cement mixture.
28. The solid cement reactant, the cement mixture or the set cement as defined in any one of items 1 to 24 for use in bone repair
29. The solid cement reactant, the cement mixture or the set cement as defined in any one of items 1 to 24 for use in bone graft substitutes.
30. The solid cement reactant, the cement mixture or the set cement as defined in any one of items 1 to 24 for use in 3D printing
31. The solid cement reactant, the cement mixture or the set cement as defined in any one of items 1 to 24 for use in preformed 3D printed implants.
32. The solid cement reactant, the cement mixture or the set cement as defined in any one of items 1 to 24 for use in coatings.
33. The solid cement reactant, the cement mixture or the set cement as defined in any one of items 1 to 24 for use in minimally invasive tissue repair surgery.
34. The solid cement reactant, the cement mixture or the set cement as defined in any one of items 1 to 24 for use in bioactive delivery.
35. A bone graft substitute comprising the solid cement reactant, the cement mixture or the set cement as defined in any one of items 1 to 24.
36. A coating comprising the solid cement reactant, the cement mixture or the set cement as defined in any one of items 1 to 24.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIGS. 1A-1D show the effect of temperature on TMPP powder; (FIG. 1A) differential scanning calorimetry and thermogravimetric analysis; (FIG. 1B) X-ray diffraction patterns as a function of temperature, Magnesium Phosphate Pentahydrate (.), Farringtonite (*); (FIG. 10) Powder density as a function of temperature; (FIG. 1D) Powder surface area as a function of temperature;

FIGS. 2A-2G show SEM micrographs of cement powders; (FIG. 2A) TMPP; (FIG. 2B) 400° C.; (FIG. 2C) 600° C.; (FIG. 2D) 700° C.; (FIG. 2E) Crystalline structure in 700° C. powder; (FIG. 2F) 800° C.; and (FIG. 2G) Crystalline structure in 800° C. powder;

FIGS. 3A-3B show the effect of heat-treatment temperature on (FIG. 3A) cement wet compressive strength and (FIG. 3B) cement phase composition, Magnesium phosphate pentahydrate (.), Farringtonite (*), when mixed with a 1.0M solution of citric acid and sodium citrate of pH 5.1 and a powder-to-liquid ratio of 1.0 g/ml;

FIGS. 4A-4B show the effect of citrate solution pH on cement initial and final setting time (FIG. 4A), and wet compressive strength (FIG. 4B) when mixed with 600° C. heat-treated powder at a powder-to-liquid ratio of 1.0 g/ml;

FIG. 5 shows the effect of citrate solution pH on phase composition, Farringtonite (*), and microstructure of magnesium phosphate cements mixed at a powder-to-liquid ratio of 1.0 g/ml after 24 hrs incubation in distilled water;

FIGS. 6A-6D show the effect of 1.0M sodium phosphate solution pH on cement initial and final setting time (FIG. 6A), and wet compressive strength (FIG. 6B), set cement porosity (FIG. 6C) and set cement phase composition (FIG. 6D), Farringtonite (*), when mixed in a powder-to-liquid ratio of 1.0 g/ml;

FIGS. 7A-7E show the effect of 1.0M sodium phosphate solution pH on the microstructure of cements mixed in a powder-to-liquid ratio of 1.0 g/ml. (FIG. 7A) pH 4.1; (FIG. 7B) pH 5.0; (FIG. 7C) pH 6.0; (FIG. 7D) pH 7.0; (FIG. 7E) pH 8.8;

FIGS. 8A-8D show the effect of citric acid weight percentage on cement initial and final setting time (FIG. 8A), wet compressive strength (FIG. 8B), set cement porosity and density (FIG. 8C) and set cement phase composition, Newberyite (°); Farringtonite (*) (FIG. 8D), when mixed in a powder-to-liquid ratio of 1.0 g/ml;

FIGS. 9A-9B show (FIG. 9A) weight loss and heat flow of magnesium phosphate cement and (FIG. 9B) the thermal analysis of the TMPP after heating at 600° C. for 30 minutes;

FIGS. 10A-10D show the effect of citric acid weight percentage on set cement microstructure. (FIG. 10A) 0 wt %, (FIG. 10B) 6 wt %, (FIG. 100) 8 wt %, and (FIG. 10D) 10 wt %;

FIG. 11 shows the cohesion of cements made with 600° C. heat-treated powder and mixed in a powder-to-liquid ratio of 1.0 g/ml;

FIG. 12 shows the injectability of the cement with citric acid addition when made with 600° C. powder at a powder-to-liquid ratio of 1.0 g/ml;

FIG. 13 shows faxitron x-ray images of magnesium phosphate (R) and brushite (L) the rabbit femurs post-retrieval (remaining cement and new bone tissue (circled)) at four weeks post-implantation;

FIGS. 14A-14B show a histological comparison of magnesium phosphate (FIG. 14A) and brushite (FIG. 14B) at four weeks post-implantation (symbols indicate: (*) remaining graft, (↑) new bone, ( [symbol] ) host bone integration);

FIGS. 15A-15B show fluorescence imaging of histological sections indicating bone formation at three weeks, (magnesium phosphate (FIG. 15A) and brushite (FIG. 15B) cements);

FIGS. 16A-16B show a light micrograph showing near complete repair of the cortical shaft of a 20 mm ulna defect after 4 weeks implantation (FIG. 16A) and a fluorescence image showing the pattern of bone formation at week 2 (FIG. 16B);

FIGS. 17A-17C show SEM images of the etched titanium rods, Untreated (FIG. 17A), Sulphuric (FIG. 17B), and Sulphuric/Peroxide (FIG. 17C);

FIGS. 18A-18C show the surface composition of the etched rods, untreated (FIG. 18A), sulphuric acid (FIG. 18B) and sulphuric/peroxide (FIG. 18C);

FIG. 19 shows the macroscale appearance of the rod coatings;

FIG. 20 shows the relationship of coating thickness to P:L with surface treatment for dip coated titanium rods;

FIG. 21 shows the relationship between cement dissolution time and P:L;

FIG. 22 shows the relative rate of cement dissolution for 0.33 g/ml cement in PBS; and

FIGS. 23A-23D show (FIG. 23A) an X-ray of explants showing dense material spanning the transverse processes; (FIG. 23B) histological examination confirming this to be bone tissue with isolated regions of material remaining visible; (FIG. 23C) a higher magnification examination showing new bone with typical osteon features formation, confirmed to occur in the first month of implantation (as shown in FIG. 23D) using fluorescent markers stained bright green.

### DETAILED DESCRIPTION OF THE INVENTION

Turning now to the invention in more details, there is provided a solid cement reactant comprising:
a dehydrated magnesium phosphate, and/or
an amorphous or partially amorphous magnesium phosphate, and/or
Farringtonite.

Herein, a “solid cement reacted” is a material, which, in the presence of a liquid cement reactant (typically an aqueous solution) will go through a cementitious reaction and thus set and harden to form a cement. The solid cement reactant may be in the form of a powder, a compressed powder, granules, or preformed blocks or components. The solid cement reactant may also be part of a composite, for example granules immobilized in a setting system, particles in a polymeric matrix, etc. wherein it may confer one or more of biological, regenerative, mechanical or handling properties.

Herein, “dehydrated magnesium phosphate” is previously hydrated magnesium phosphate that has been at least partially dehydrated. The dehydration does not need to be complete, it is sufficient that at least part of the water molecule hydrating the starting material is removed. In embodiments, the dehydration removed about 10% or more of the water, for example 20%, 40%, 60%, or 80% or more. In embodiments, the dehydration is complete. The dehydration can be performed by any usual means known to the skilled person, for example heating. Dehydration of hydrated salts may also be effected by non-thermal means such as preparation in partially or completely anhydrous conditions, e.g. alcoholic precipitation, storage under vacuum and other such methods known to those skilled in the art. The cement forming ability of the dehydrated magnesium phosphates can be assessed by reaction with an aqueous liquid to form a hardened paste.

Partially amorphous magnesium phosphate is magnesium phosphate that is not totally crystalline or contains an amorphous fraction. The amorphous nature of the magnesium phosphate can be confirmed by the usual techniques knows to the skilled person. These techniques include a reduction of the intensity of X-Ray diffraction peaks, recrystallisation as determined by thermal analysis such as TGA or reactivity or high solubility in aqueous liquids e.g. to form a cement.

Amorphous magnesium phosphates and cement forming magnesium phosphates may be produced by a number of techniques as well as dehydration of magnesium salts. Mechanical activation and direct precipitation of amorphous magnesium phosphates (with or without stabilizers such as pyrophosphate, manganese ions and so forth) are well known methods amongst those skilled in the art. In embodiments, the amorphous or partially amorphous magnesium phosphate is obtained by heat-treatment of a magnesium phosphate, for example magnesium phosphate pentahydrate (Mg₃(PO₄)₂.5H₂O). In embodiments, the heat treatment is heating at a temperature between 400 and 800° C., preferably 600° C., for about 30 minutes. The skilled person will know how to vary the heating temperature, time and calcinations atmosphere and pressure depending on the starting material heating rate and other relevant factors.

In embodiment, the solid cement reactant can be a dehydrated magnesium phosphate that is also amorphous or partially amorphous.

The present invention also relates to a cement mixture obtained by mixing the solid cement reactant with a liquid cement reactant. The invention also relates to a set cement obtained upon setting of this cement mixture.

In embodiment, the liquid cement reactant is an aqueous solution. In embodiments, it contains organic acid ions, such as citrate, and/or monovalent cations, such as sodium ions, and/or phosphate ions. In embodiments, the liquid cement reactant is a buffer solution. In embodiments, the liquid cement reactant has a pH between 1 and 11, preferably between 3 and 10, more preferably between 4 and 9, and even more preferably between 4 and 8 (prior to mixing with the solid cement reactant).

In embodiments, the solid cement reactant may further comprise an organic acid or a salt thereof. In embodiment, the organic acid or salt thereof is citric acid or a citrate salt, such a sodium citrate. In embodiments, the solid cement reactant comprises a soluble salt in a quantity that would be sufficient to produce aqueous solution with a pH between about 3 and about 9 in a amount corresponding to the amount of liquid cement reactant intended to the used with the solid cement reactant. In this embodiment, in effect, the soluble salt that would be present in the liquid cement reactant is provided in the solid cement reactant instead.

In embodiments, the solid cement reactant comprises citric acid, for example in a concentration ranging between about 2 and about 20 wt % based on the total weight of the solid cement reactant.

In embodiments, the liquid cement reactant is a citrate solution, for example a sodium citrate solution. In embodiments, its pH is between 4 and 8. In embodiments, its pH is 5.1. In embodiments, the liquid cement reactant is a phosphate solution, for example a sodium phosphate solution. In embodiments, its pH is between 4 and 8. In embodiments, its pH is 7. In embodiments, the liquid cement reactant comprises citrate and phosphate ions.

Other non-limiting examples of organic acids for inclusion in liquid or solid cement reactants include fumarates, tartrates, glycolates, etc.

Compounds known to the skilled person to act as accelerators, retardants, and/or viscosity reductants in cements, including for example polyanions, silicates and so forth, can also be used in the liquid and/or solid cement reactants.

The cement reactants can also be seeded with cement products that will act as accelerators.

In embodiment, the cement produced by the setting of a mixture of the solid cement reactant with the liquid cement reactant has a crystalline phase that is predominantly Farringtonite or has crystalline phases that are predominantly Farringtonite and Newberyite. Herein, “Farringtonite” refers to Mg₃(PO₄)₂ and “Newberyite” refers to Mg(PO₃OH).3(H₂O). In embodiments, the cement displays an exothermic peak between 600-700° C., when analyzed by thermal analysis.

In embodiments, the mixture of the solid cement reactant with the liquid cement reactant comprises an amorphous magnesium phosphate, alkali metal ions and an aqueous solution,

The present invention also relates to a kit comprising the above solid cement reactant. In addition, the kit may comprise instructions for using the solid cement reactant to effect a setting reaction and/or a liquid cement reactant or a component to be mixed with water or an aqueous liquid to form a liquid cement reactant, and/or one or more devices for mixing reactants or for the delivery or application of the cement mixture.

The above liquid and solid cement reactants and cement have various applications. They can be used in bone repairs, for example as bone graft substitutes. They can be used for 3D printing, for example as preformed 3D printed implants. They also can be used as coatings. They can also be used for minimally invasive tissue repair surgery and for bioactive delivery.

Therefore, the present invention also relates to bone graft substitutes comprising the above cement or the above solid cement reactant; to coatings comprising the above cement, and to substrates, such as orthopaedic implants, comprising such a coating.

In the case of printed components, the solid cement reactant might simply be bound together rather than reacted through a cementitious reaction so that the component may be handled. Setting may then occur in the animal or patient or a separate curing process may occur. Alternatively the setting may occur partially or fully during printing,

In embodiments, the cement of the invention is osteoconductive, has appropriate setting time and compressive strength for use in bone repair, is injectable and has a predictable dissolution time and is cohesive. For this application, the cement slurry (i.e. the mixture of the solid cement reactant with the liquid cement reactant) powder-to-liquid ratio (P:L) can vary from about 0.2 g/ml to about 5 g/mL.

As stated above, the present invention also relates to the use of the above cement as a coating. In embodiments, the cement can be dip-coated onto, for example, titanium rods. It can therefore be used for coating, for example orthopaedic implants. Coating of titanium rods can be performed using a simple dip coating method. The coating thickness can be controlled by the cement slurry powder-to-liquid ratio (P:L) with lower ratios resulting in thinner coatings. For this application, the cement slurry powder-to-liquid ratio (P:L) can vary from about 0.1 g/ml to about 2 g/mL.

Other objects, advantages and features of the present invention will become more apparent upon reading of the following non-restrictive description of specific embodiments thereof, given by way of example only with reference to the accompanying drawings.

### Description of Illustrative Embodiments

The present invention is illustrated in further details by the following non-limiting examples.

Below, the synthesis, optimization and adaptation of a novel magnesium phosphate bone cement derived from magnesium phosphate pentahydrate is presented. The inventors determined that heating of the magnesium phosphate pentahydrate induced a crystallographic transformation into a more amorphous material which can be used as a cement reactant.

The material was mixed with a citrate solution and set to form a ceramic. In a particular instance, a magnesium phosphate cement was synthesized using a pH 5.1 citrate solution.

A strong material was obtained using powder heated to 500 or 600° C. for 30 minutes mixed with a pH 7.0 sodium phosphate solution yielding compressive strengths upwards of 22 MPa, which is comparable to the compressive strength of cancellous bone (1.5-9.3 MPa). The effect of sodium phosphate solutions on the cements as investigated. A sodium phosphate solution of pH 7.0 was found to reduce the setting time while simultaneously increasing the compressive strength to 22 MPa. The setting time was further decreased with the addition of citric acid crystals to the solid phase of the cement, reducing the setting time to 15 minutes at 8 wt % addition. In addition, the citric acid also helped to improve the injectability of the cement, from 22% to 80% injectability.

A measure of biocompatibility came in the form of in vivo data.

The magnesium phosphate cement using pH 7.0 sodium phosphate was also adapted for coating orthopaedic implants and drug release. Coating of titanium rods was performed using a simple dip coating method. Coating thickness was controlled by the cement slurry powder-to-liquid ratio (P:L) with lower ratios resulting in thinner coatings. Dissolution of cement pellets at each P:L ratio had controlled dissolution ranging from 80-110 days to complete dissolution.

### Trimagnesium Phosphate Cement for Biomedical Applications

The inventors examined the properties of amorphous magnesium phosphate and its ability to form a cement. The material was characterized using x-ray diffraction, differential scanning calorimetry, thermogravimetric analysis, helium pycnometry and scanning electron microscopy to determine phase changes with heating, crystalline composition, material density and morphology. Heat-treated powder was mixed with setting liquids of various pH to determine the optimal setting time, compressive strength and composition. The biocompatibility of the material was examined using mouse pre-osteoblast cells to determine cell toxicity.

The inventors determined that the mixing of citrate solutions with heat-treated trimagnesium phosphate pentahydrate (TMPP) results in a strong, biocompatible cement. Heat-treatment of the TMPP at 600° C. yielded a mostly amorphous material which, when mixed with pH 5.1 citrate solution, formed a solid with a wet compressive strength of 19.1 MPa.

Materials and Methods

Materials

Trimagnesium phosphate pentahydrate (Mg₃(PO₄)₂.5H₂O; TMPP) powder was obtained from Jost Chemical (St. Louis, Mo., USA) and citric acid (CA) and sodium citrate (SC) were both obtained from Fisher Scientific (Ottawa, ON, Canada).

Cement Powder Synthesis and Characterisation

Thermogravimetric analysis (TGA) and differential scanning calorimetry (DSC) analysis of TMPP powder was performed using an SDT Q600 (TA Instruments, New Castle, Del., USA). Using the information from the curves, the powder was heated to various temperatures for 30 minutes to create powders with varied amorphous and hydrated states.

X-ray diffraction (XRD) analysis of the powders and cement was performed to evaluate their crystallographic nature and conduct phase analysis. A vertical-goniometer X-ray diffractometer (Philips model PW1710, Bedrijven b. v. S&I, The Netherlands), equipped with a Cu Kₐ radiation source, was used for the powder diffraction pattern collection. Data was collected from 20 of 5 to 55° with a step size of 0.03° and a normalized count time of 1.5 s per step. The phase composition was examined using reference patterns (00-035-0329, 00-019-0767, 00-011-0235) from the International Centre for Diffraction Data (TODD). The morphology of the treated powders was examined using scanning electron microscopy (SEM; JEOL JSM-840A operating at 15 kV). Powder specific surface area was measured using BET adsorption of nitrogen (TriStar, Micromeritics Instrument Corporation, Norcross, Ga., USA).

Cement Preparation

The heat-treated powders were mixed with solutions of 1.0M citric acid and 1.0M trisodium citrate mixed in various ratios, as presented in Table 3.2. All cements were mixed with a powder-to-liquid ratio (P:L) of 1.0 g/ml. Immediately after mixing, the cement paste was cast into cylindrical specimens (6 mm ø×12 mm), using a PTFE split mould. Cement setting time was measured using Gillmore needles (ASTM C266-08). After final setting was reached, the cement cylinders were removed from the mould and immersed in distilled water at 37±1 ° C. and 100% relative humidity for 24 hours before further analysis.

TABLE 3.2
Citric acid and Sodium Citrate Solutions

| CA:SC Ratio | pH |
|---|---|
| 1:0 | 2.2 |
| 2:1 | 3.9 |
| 1:1 | 4.4 |
| 1:2 | 5.1 |
| 0:1 | 7.8 |

Cement Analyses

Following 24 hours incubation, the cement cylinders were removed from the distilled water and patted with a damp paper towel to remove any extraneous surface water before being weighed. Cement strength was tested using a universal testing machine (Instron 5569, Norwood, Mass., USA) with a crosshead speed of 0.1 mm/min. The resultant pieces were dried at 30° C. under vacuum.

Following drying, the cement pieces were weighed and apparent density (pa) was calculated using the initial formed dimensions. The real density (ρr) of the cements was measured using helium pycnometry (AccuPyc 1330, Micromeritics Instrument Corporation, Norcross, Ga., USA). Using the two density values, the percentage porosity was calculated using the following equation:

$$Porosity ( \% ) = ( 1 - \frac{{ρ}_{a}}{{ρ}_{r}} )$$

Characterization of the cement morphology was performed using SEM. Surface area of the cements was measured using BET adsorption.

Results

Cement Powder Characterisation

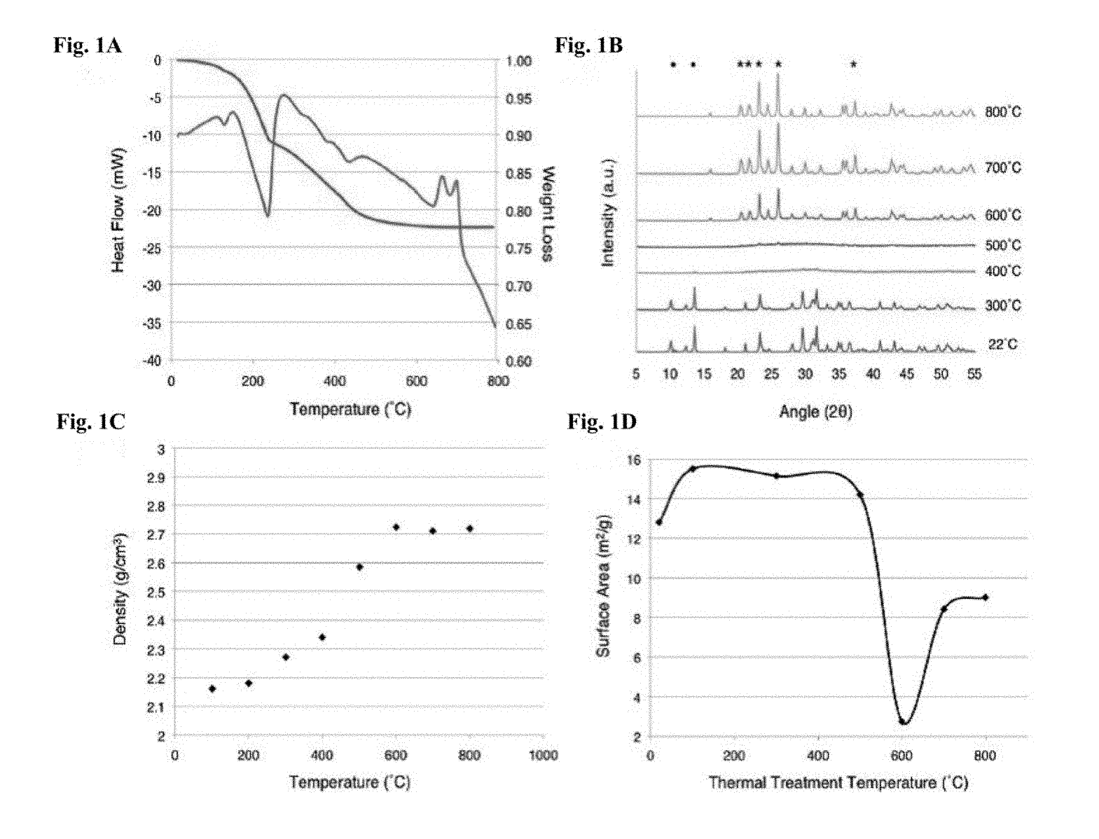

Thermal analysis of the TMPP powder revealed changes in chemical structure as temperature increased. The DSC data (FIG. 1A) showed a large significant endothermic event around 230° C. followed by an exothermic event peaking around 300° C. The TGA showed a three-stage weight loss, with a change in the slope occurring at 230° C. corresponding to the endothermic event in the DSC pattern. Dehydration and transformation from the hydrated crystalline to amorphous state and finally to Farringtonite, as determined by XRD, is reflected in the TGA curve as final crystallization occurs at temperatures after the loss of crystal water.

XRD of the TMPP powder at ascending temperatures revealed transformation from a crystalline structure to an amorphous state followed by a transformation into a crystalline material (FIG. 1B). The material remained as crystalline TMPP upon heating to 300° C. Further heating induced a change from a crystalline to amorphous structure, present from 400-500° C. Above 500° C. the induction of crystallization occurs, with peaks of Farringtonite (Mg₃(PO₄)₂) beginning to show at 600° C. The Farringtonite peaks became more pronounced when heated to 700° C. and 800° C.,

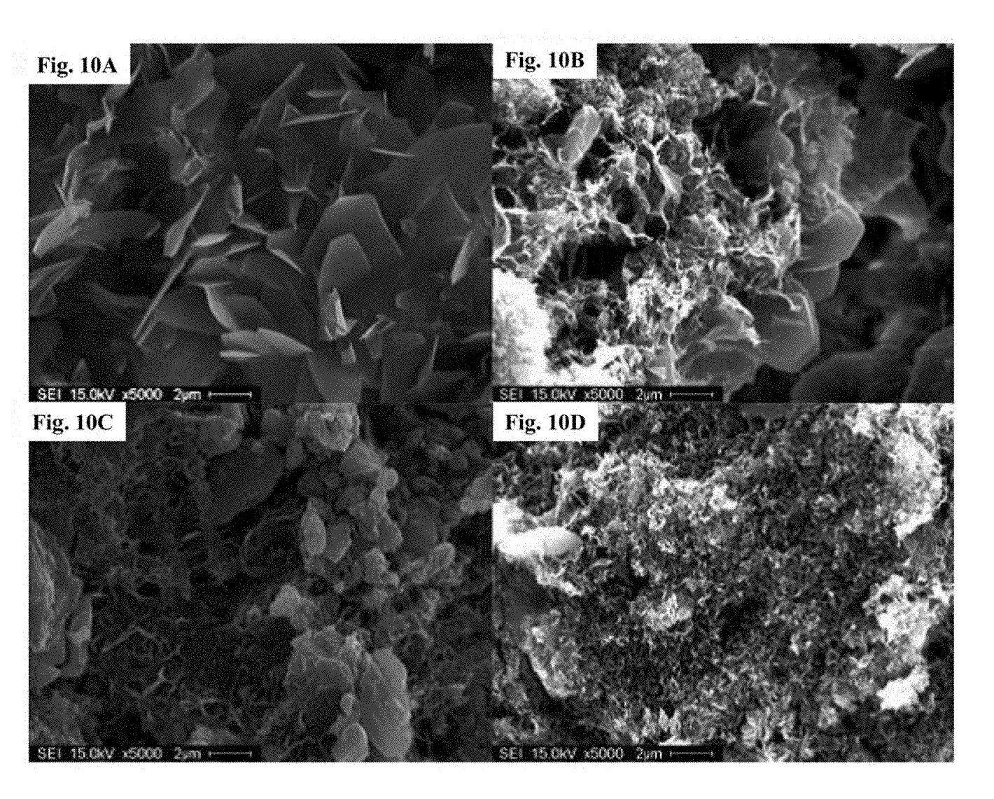

The loss of crystalline water was also reflected in the change in powder density (FIG. 10) that occurred as heat treatment temperature increased. The loss of the water from the crystal structure resulted in the shrinking of the lattice size and increased powder density.

The surface area of the treated powders (FIG. 1D) initially increased with heat treatment, remaining relatively constant until 600° C. where a large drop occurred. Upon further heating, the surface area reduced to just over half the initial surface area of the TMPP powder.

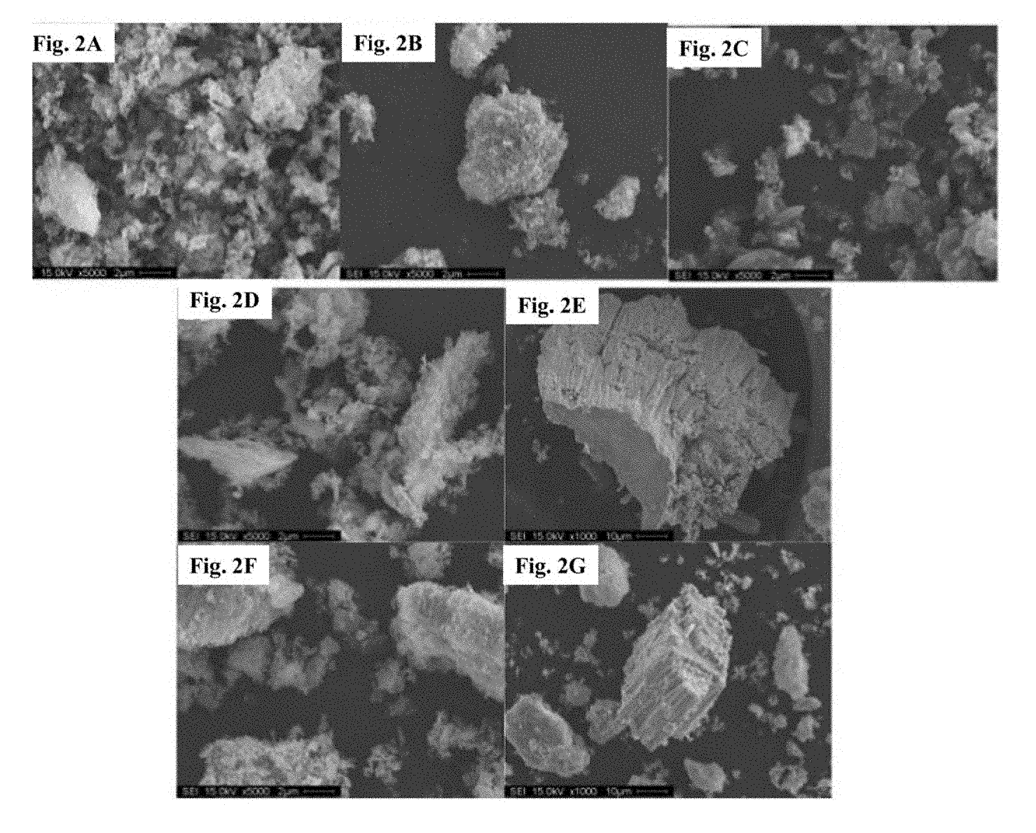

SEM micrographs (FIGS. 2A-2G) revealed the starting TMPP powder consisted of agglomerates composed of smaller crystals. As the treatment temperature of the powder increased, the agglomerates remained, showing no evidence of a morphological change to a glassy structure. At 600° C., small crystal-like structures can be seen mixed in agglomerates. As the powder was heated to 700° C. and 800° C., the size of the crystals present increased greatly showing a clear growth orientation, though agglomerates were still present.

Cement Characterisation

Effect of Powder Thermal Treatment

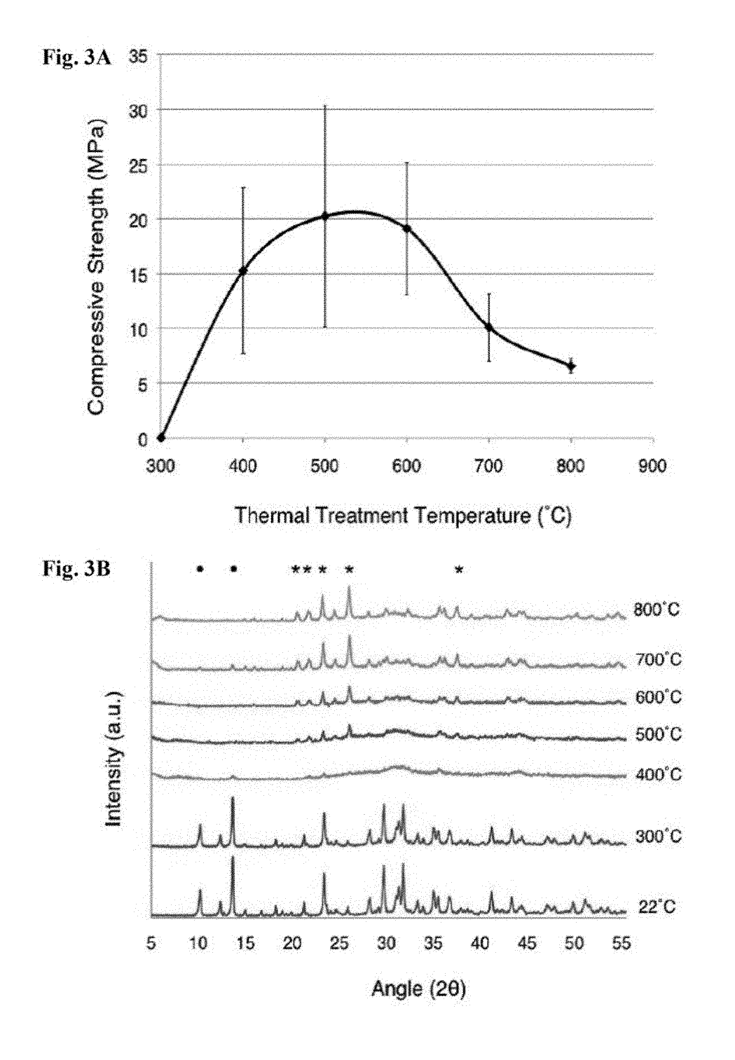

The heat-treated powders were mixed with a pH 5.1 solution of citric acid and sodium citrate, which yielded a range of cement strengths as seen in FIG. 3A. Both the TMPP and 300° C. powder failed to set after being allowed to cure for several days. The highest strength cements were formed with the 500° C. and 600° C. powders. Both cements showed similar average strengths of 20.2 and 19.1 MPa, respectively. Though the cements have similar strengths, the 600° C. powder was chosen for all subsequent experiments. Cement made with 700° C. powder resulted in a product with half the strength of the 500° C./600° C. cements. Although the predominantly crystalline 800° C. powder formed a viable cement, it required 2 days to reach final setting strength.

XRD patterns of the set cements (FIG. 3B) followed a similar trend to that seen in the heat-treated powders (FIG. 1B). The crystallinity of the product was largely unchanged from that seen in the powders prior to mixing with the citrate solution, indicating cement setting does not rely on a crystallization reaction. Crystallinity of the 400° C. and 500° C. cements was greater compared to the previous powder patterns, with small, more defined regions appearing.

Effect of Citrate Solution pH

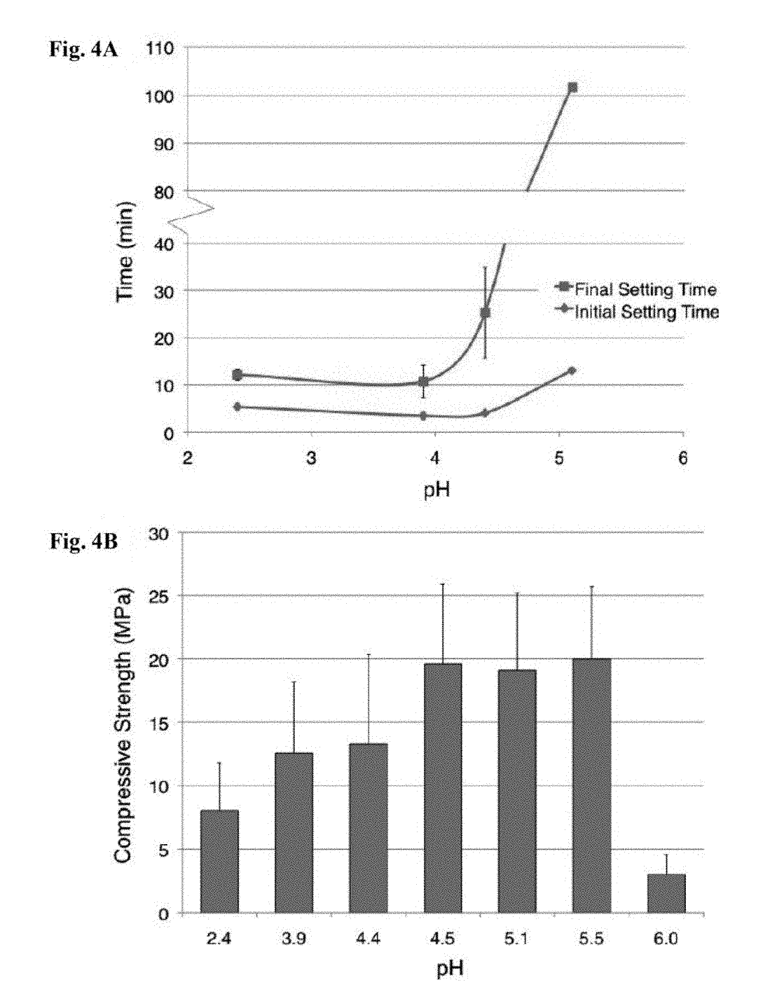

Mixing of the 600° C. powder with citrate solutions of various pH yielded vastly different setting times. Mixing the 600° C. powder with 1M citric acid caused a rapid reaction resulting in a mixture with a gummy texture which was difficult to apply to the mould and measure the initial setting time of. Citric acid was then mixed with sodium citrate in various ratios to yield solutions that would yield slower initial setting times. Mixing each of the cement powders, the most rapid setting time was obtained using a solution of pH 3.9. Setting times appeared to increase considerably as pH increased with a final setting times of almost 2 hours required for the pH 5.1 solution (FIG. 4A) and solutions of higher pH showing no initial setting strength following 2 h cure. The relationship between solution pH and compressive strength differs, the cements having more rapid setting times were found to be weakest, with increasing pH resulting in stronger cements. This result continues until a plateau between pH 4.5-5.5 where the strength is at its highest (FIG. 4B). Further increases in pH resulted in weaker cements followed by mixtures that no longer cured.

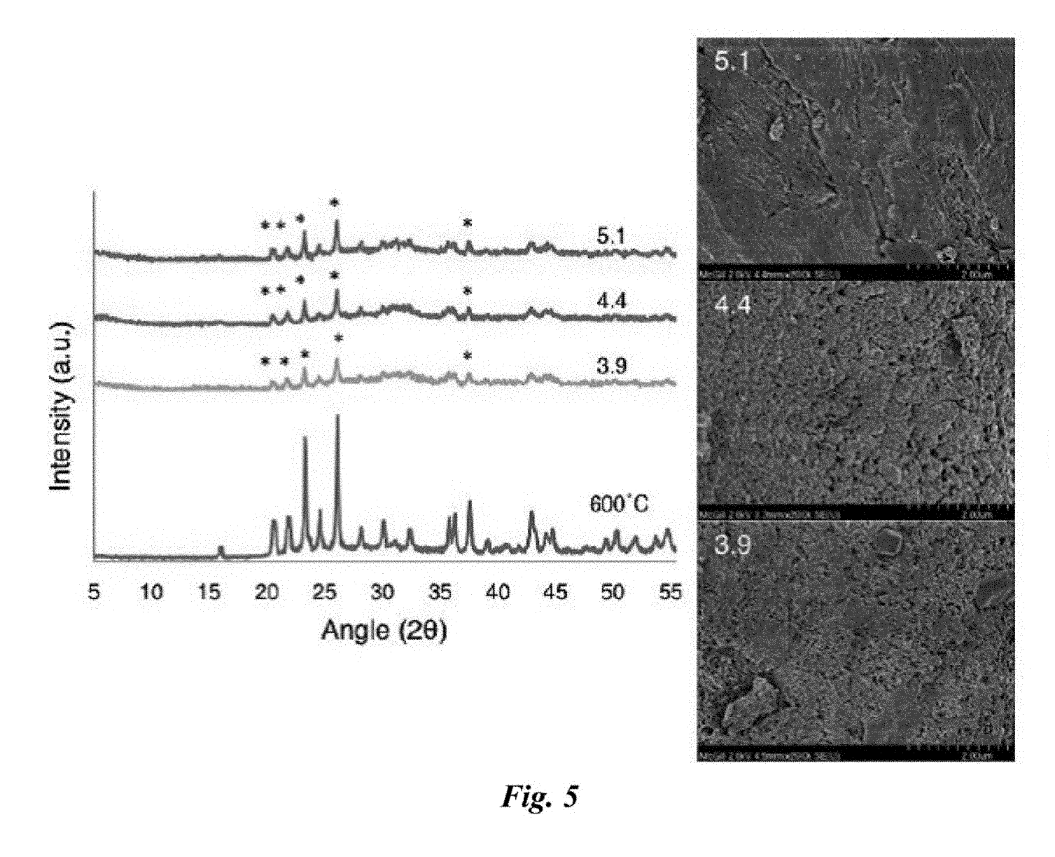

XRD analysis of cements created with pH 3.9, 4.4 and 5.1 solutions revealed closely related diffraction patterns. While the product remained largely amorphous, the crystalline phase present was composed of Farringtonite (FIG. 5). Differences in cement morphology were observed upon SEM imaging. Cement compositions made with pH 3.9 and 4.4 solutions showed a more porous surface whereas the pH 5.1 solution showed a relatively uniform surface.

Measurement of cement porosity using BET (Table 3.3) revealed that surface area increased with solution pH. The relationship between solution pH and porosity was inverse, with the lower pH solution resulting in the highest percentage of porosity.

TABLE 3.3
The effect of citrate solution pH on surface area and porosity
of cements mixed at a powder-to-liquid ratio of 1.0 g/ml after
24 hrs incubation in distilled water.

| pH | BET Surface Area (m²/g) | Porosity (%) |
|---|---|---|
| 5.1 | 5.2 ± 0.01 | 21.7 ± 1.8 |
| 4.4 | 4.1 ± 0.01 | 35.9 ± 0.5 |
| 3.9 | 3.2 ± 0.01 | 39.0 ± 2.5 |

Discussion

Although TMPP itself does not react to form cement, the inventors discovered it was possible to make self-setting cements from heat-treated TMPP. The ability of the heat-treated powders to create viable cement showed a distinct trend with increasing compressive strength. The ability of the powder to produce a cement increased with the amorphousness of the material. The highest strength cements produced with a pH 5.1 citrate solution were made using the 500° C. and 600° C. powder. The XRD patterns of the cements show that mixing of the powders treated outside the amorphous range show no considerable change in their crystallinity, while the powders from 400-600° C. showed small crystal peaks. This is more evident in the comparison provided in FIGS. 3A-3B, as the amorphous powders upon reaction with the liquid phase appear to approach an equilibrium between amorphous and crystalline phases.

To the inventor's knowledge, an amorphous-crystalline magnesium phosphate cement composed of a Farringtonite or Farringtonite and Newberyite crystalline phase has not been reported previously. The chemical composition of the cement is expected to provide good resorbability with Farringtonite having a solubility similar to that of tricalcium phosphate (TCP). The cement of the invention has synthesis advantages over TCP, as synthesis occurs at room temperature, it can be applied to defects as a wet mix curing in situ, whereas there are no TCP cements due to the high temperature synthesis required.

Citric acid allowed the cement to set and form the final product and mixing of the heat-treated powders with water did not yield a set product.

The effects of the citrate solution on cement setting were related to solution pH. Low pH solutions resulted in rapid setting cements with low strength. Increases in solution pH resulted in slower setting reactions and increasing strength. The relationship between the setting time and the strength begins to diverge around pH 4.5 where strength begins to plateau while the setting time continues to increase. The strength of the cement showed a plateau from pH 4.5-5.5 before dropping off sharply. The lack of plateau in setting time with increasing pH suggests cement strength is not directly related to setting time. The differences in cement properties due to pH were not reflected in the XRD profiles. The cements mixed at various pH showed nearly identical profiles (FIG. 5), indicating solution pH is a factor in the strength and setting times, but not in the crystalline composition of the cements.

The cement formulation produced using 600° C. heat-treated cement powder with a pH 5.1 citrate solution possesses strength 3 times that of cancellous bone (6 MPa) and 10-60% of the strength of cortical bone based on loading direction. The similar strength of the material to that of cancellous bone provides the opportunity to replace materials previously used in the repair of cancellous bone and non-load bearing applications.

The cement we have presented represents a new class of calcium-free bone cement which is able to set in ambient conditions to form an amorphous-crystalline trimagnesium phosphate solid. With its strength similar to that of cancellous bone, and excellent biocompatibility our cement could provide a viable alternative to current calcium phosphate cements.

Investigation into the Optimization of the Magnesium Phosphate Cement System

The inventors optimized and enhanced the properties of the cement system discussed in the section entitled “TRIMAGNESIUM PHOSPHATE CEMENT FOR BIOMEDICAL APPLICATIONS” above by investigating changes in raw material composition and the use of additives. The effect of these changes was determined through measurement of the setting time, mechanical strength, cohesion, porosity, microstructure, injectability and cement biocompatibility.

Herein, the inventors demonstrate modification of this magnesium phosphate cement system by using sodium phosphate solutions and citric acid crystals. The use of a pH 7.0 sodium phosphate solution increased the strength of the cement material to 22.6 MPa. The use of 8wt % citric acid crystals in the cement powder phase allowed for a reduction in cement setting time from 44 min to 15 min. Investigation into the cement's ability to regulate cell function showed the steady up-regulation of five factors know to be important to osteoblast differentiation and bone formation.

Materials and Methods

Materials

Trimagnesium phosphate pentahydrate (Mg₃(PO₄)₂.5H₂O; TMPP) powder was obtained from Jost Chemical (St. Louis, Mo., USA), citric acid (CA) and sodium phosphate monobasic were obtained from Fisher Scientific (Ottawa, ON, Canada) and sodium phosphate dibasic was obtained from Sigma-Aldrich (Oakville, ON, Canada).

Cement Powder Synthesis

The cement powder was synthesized as previously reported by us in the section entitled “TRIMAGNESIUM PHOSPHATE CEMENT FOR BIOMEDICAL APPLICATIONS” above. Only powder heat-treated at 600° C. was used in this study.

Cement Preparation

Cement powder was mixed with solutions of various pH made from 1.0M solutions of sodium phosphate monobasic and sodium phosphate dibasic. All cements were mixed at a powder-to-liquid ratio (P:L) of 1.0 g/ml. Immediately after mixing, the cement paste was cast into cylindrical specimens (6 mm ø×12 mm) using a PTFE split mould. Cement setting time was measured using Gillmore needles [ASTM standard C266]. After final setting was reached, the cement cylinders were removed from the mould and incubated in distilled water at 37±1 ° C. and 100% relative humidity for 24 hours before further analysis.

Cement Analysis

Following 24 hours incubation, the cement cylinders were removed from the distilled water and patted with a damp paper towel to remove any extraneous surface water. The cylinders were weighed and measured for length. Wet compressive strength of each cylinder was tested using a universal testing machine (Instron 5569, Norwood, Mass., USA) with a crosshead speed of 0.1 mm/min. The resultant pieces were dried at 30° C. under vacuum.

Following drying, the cement pieces were weighed and the apparent density (pa) was calculated using the initial formed dimensions and the real density (ρr) of the cements was measured using helium pycnometry (AccuPyc 1330, Micromeritics Instrument Corporation, Norcross, Ga., USA). Using the two density values, the percentage porosity was calculated using the following equation:

$$Porosity ( \% ) = ( 1 - \frac{{ρ}_{a}}{{ρ}_{r}} )$$

Characterization of the cement morphology was performed using scanning electron microscopy (SEM; JSM-840a, JEOL Corporation, Japan).

Cement Setting Time Optimization

Setting time of the cement was optimized through the addition of anhydrous citric acid crystals in various weight percentages to the cement powder. The cements were made using a powder-to-liquid ratio of 1.0 g/ml. Both setting time and wet compressive strength were measured for the modified cements. Cement phase composition was determined using XRD and cement microstructure was examined using SEM.

Cement Cohesion

Cohesion of the cement was determined by measuring particle release as was previously described (Alkhraisat 2008). Briefly, Cement tablets (7.63 mm ø×3.90 mm) were made using microfuge tube caps. The caps were loaded with cement immediately following mixing and placed open-side down in distilled water. After 24 hours, the caps were removed from the water and the tablets removed from the caps. Filter paper (Fisherbrand Quantitative Q2 filter discs, Fisher Scientific, Ottawa, ON, Canada) was used to filter non-cohesive particles from the water. Following filtration, the tablets and filter paper were dried at 30° C. under vacuum. Once dry, the tablets and filter paper were weighed. The cohesion of the cement was determined using the mass of the tablets (mt) and the mass of the release particles (mr) with the following equation:

$$Cohesion ( \% ) = ( \frac{{m}_{t}}{{m}_{t} + {m}_{r}} ) \times 100 \%$$

Cement Injectability

Cement injectability was investigated with various citric acid weight percentages. The cements were mixed at a powder-to-liquid ratio of 1.0 g/ml and loaded into 5 cc syringes. Following loading, the syringe and cement contents were weighed (mi). The cement was then injected using moderate hand pressure. Following injection, the syringe was weighed to determine the mass of the remaining cement (mr). Injectability was expressed using the following equation:

$$Injectability ( \% ) = ( \frac{{m}_{i} + {m}_{r}}{{m}_{i}} ) \times 100 \%$$

Results

Effect of Sodium Phosphate Solution pH

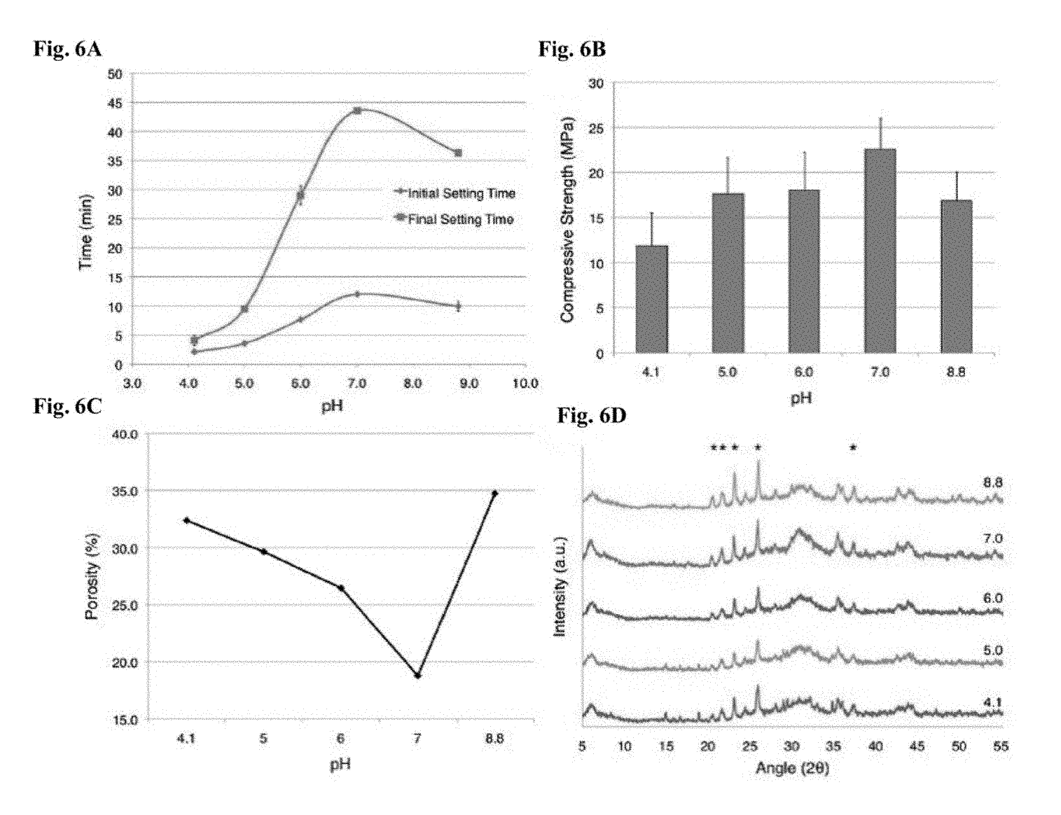

Mixing of the powder with each of the sodium phosphate solutions yielded pastes which all set to form cements. As pH of the solutions increased, so did cement setting times. A maximum time was reached at pH 7.0, before decreasing toward pH 9.0 (FIG. 6A). The pH-setting time relationship was reflected in the wet compressive strength of the set cements. Strength increased with increasing pH until pH 7.0 where a maximum strength of 22.6 MPa was reached before decreasing with further increases in solution pH (FIG. 6B).

Cement porosity followed an inverse relationship to that of the setting times and strength (FIG. 6C). Decreases in porosity occurred with increasing solution pH. At pH 7.0, a minimum porosity of 18.77% was reached after which the porosity increased to its maximum of 34.73% at pH 8.8.

Changes in pH of the sodium phosphate solution showed few differences in the set cement phase composition. XRD patterns as compared to the starting powder (FIG. 6D) show set cements formed products less crystalline than the starting powder. All cements, regardless of the pH, showed similar patterns with many of the Farringtonite peaks (*) from the starting powder preserved, though of lesser intensity.

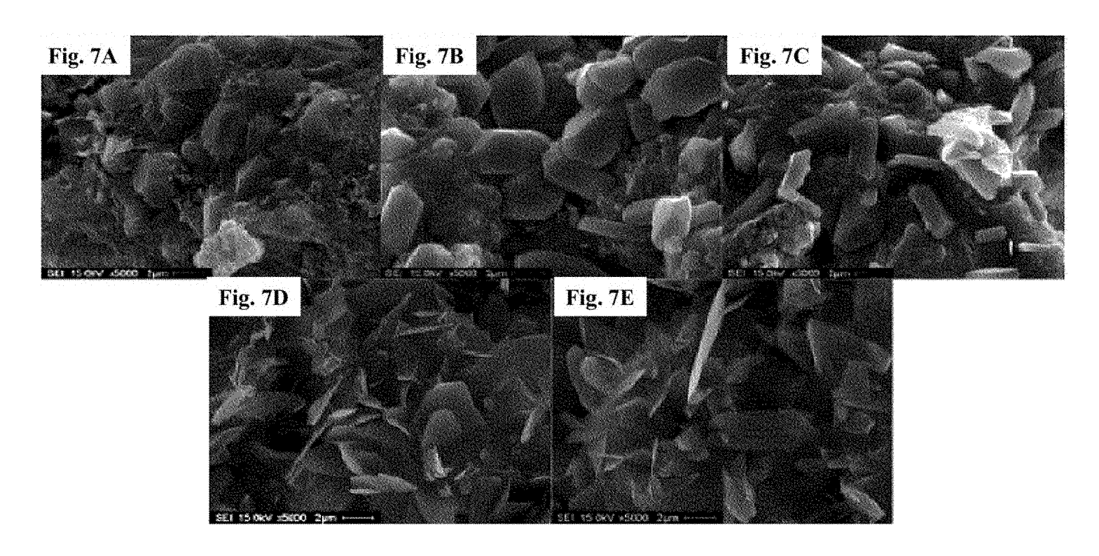

SEM of the set cements revealed a microstructure of small crystallites held together by an amorphous phase. Crystallite morphology and size was relatively common between cements made with solutions from pH 4.1-6.0 (FIGS. 7A-C), with crystallites 3-4 μm in length. Cement made with pH 7.0 solution showed very different crystallite morphology. Crystallites were thin blade-like structures of similar length to crystallites of lower pH cements. Cement made with pH 8.8 sodium phosphate resulted in same blade-like structures.

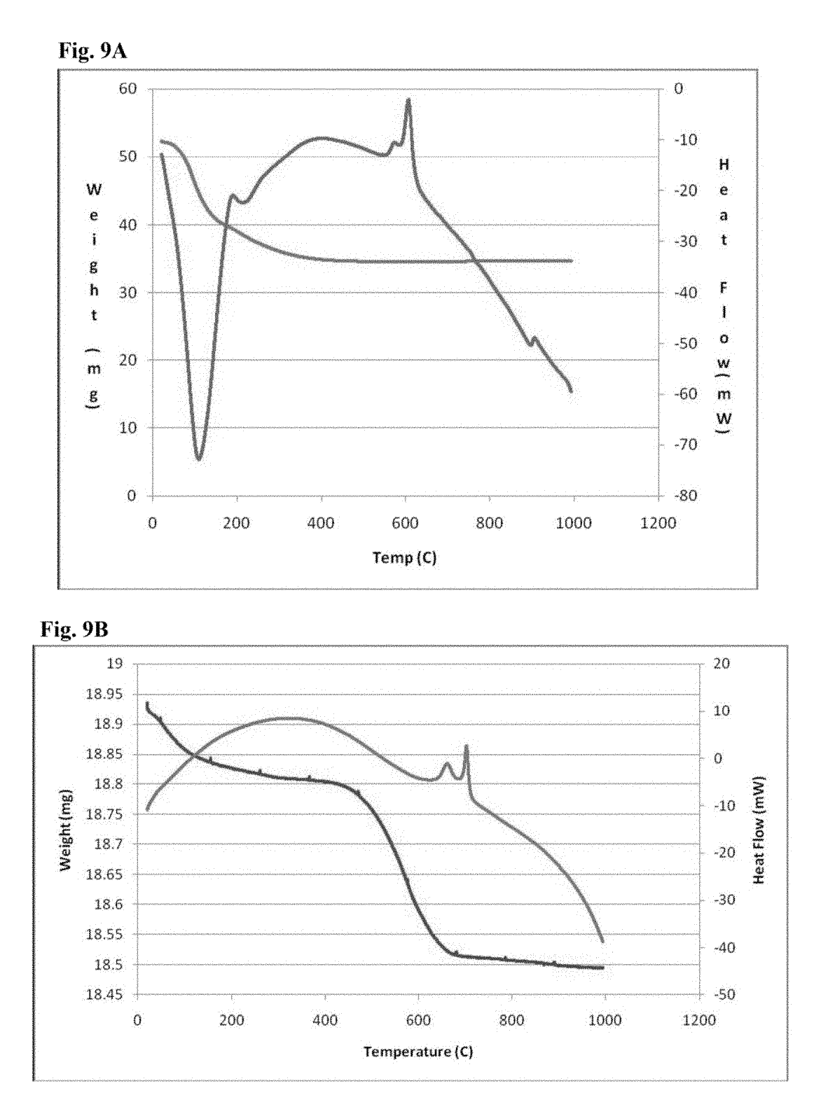

Thermal characterization of this set cement made in FIG. 9A. The curve showing the dip at a temperature of about 100° C. shows the heat flow, the other curve shows the weight loss. It has been determined that the set cement was approximately a third to fifth water and shows exothermic peaks between 600 and 700 C.

FIG. 9B shows the thermal analysis of the TMPP after heating at 600° C. for 30 minutes. A 2% weight loss to 700° C., mainly between 500 and 600° C. and exothermic events between 700 and 800° C. can clearly be seen.

Setting Time Optimization Using Citrate

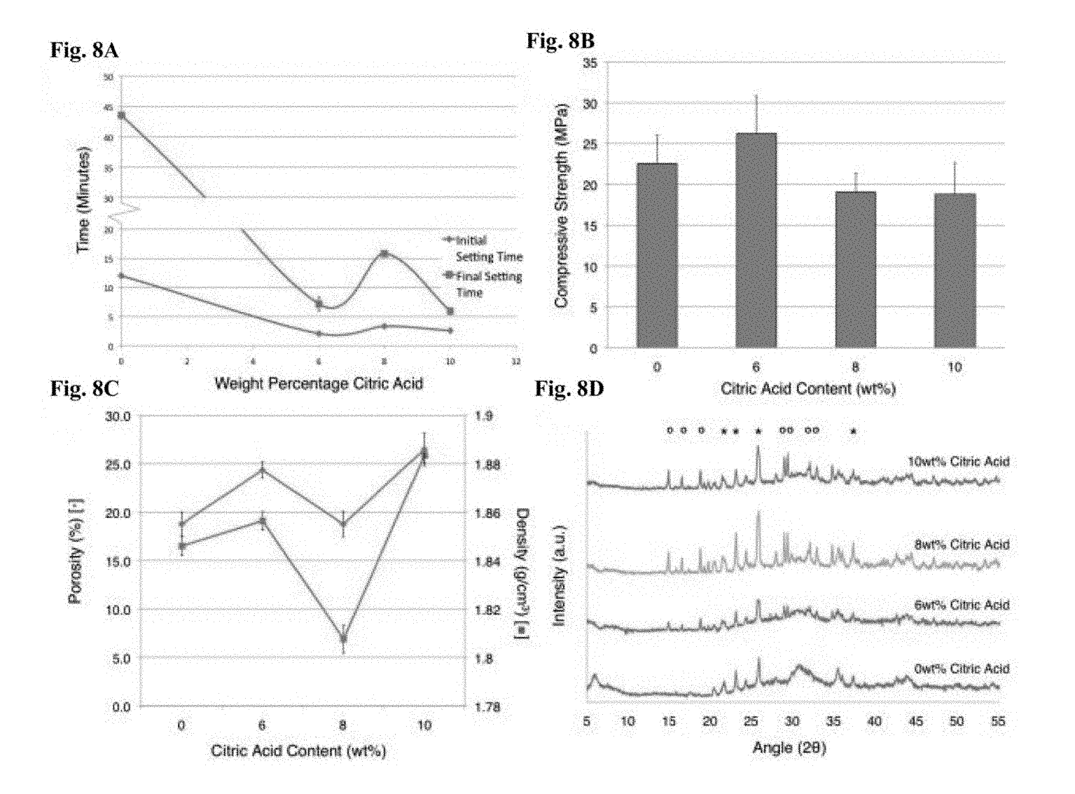

The addition of ground anhydrous citric acid crystals to the cement powder resulted in large decreases in the setting time of the cements when mixed with pH 7.0 sodium phosphate solution (FIG. 8A). The cement set rapidly with the addition of 6wt % CA, with a final setting time of approximately 7 minutes. Further addition to 8wt % CA resulted in slightly slower setting times, with final setting occurring at approximately 16 minutes. An increase in CA content to 10wt % shortened the setting time to a rapid final setting time of 6 min.

Citric acid content in the powder had a direct effect on the strength of the cements (FIG. 8B). Cements made with 6 wt % CA showed an increase the set cement strength, with an average strength of 26 MPa. The additions of 8 wt % and 10 wt % resulted in decreases in strength with both approximately 19 MPa in strength.

Examination of the density of the cements with the addition of citric acid showed slight increases with increases in CA added, as seen in FIG. 8C. Citric acid content did not show a consistent effect on porosity as 6 wt % showed an increase, while 8 wt % resulted in porosity close to that of 0 wt %. The porosity of the 10 wt % composition increased above the level of the 6 wt % cement to just over 25%.

XRD phase analysis revealed the addition of citric acid to the cement powder resulted in the formation of a newberyite phase in the cement (FIG. 8D). Cements formed with 6 wt % CA showed moderate newberyite peaks along with Farringtonite peaks found in the cement powder and non-citric cements. The cements with 8 wt % and 10 wt % CA showed more defined newberyite and Farringtonite peaks.

In clinical applications, cement setting time must be rapid enough to ensure the cement provides support shortly after the procedure, yet not too rapid to allow the surgeon time to ensure the site of implantation is adequately filled. Therefore, based on these requirements, cements made using 8 wt % CA were used for further experiments.

SEM of the cements with the addition of citric acid showed vastly different microstructures to that of the cement without (FIGS. 10A-10D). The blade-like structures of the cement were not seen, but instead crystallites were more regular shaped, surrounded by a web-like network. The tightness of the network increased with citric acid content. The microstructure of the citric added cements more closely resembled the structure of the cement made with pH 4.1 sodium phosphate solution.

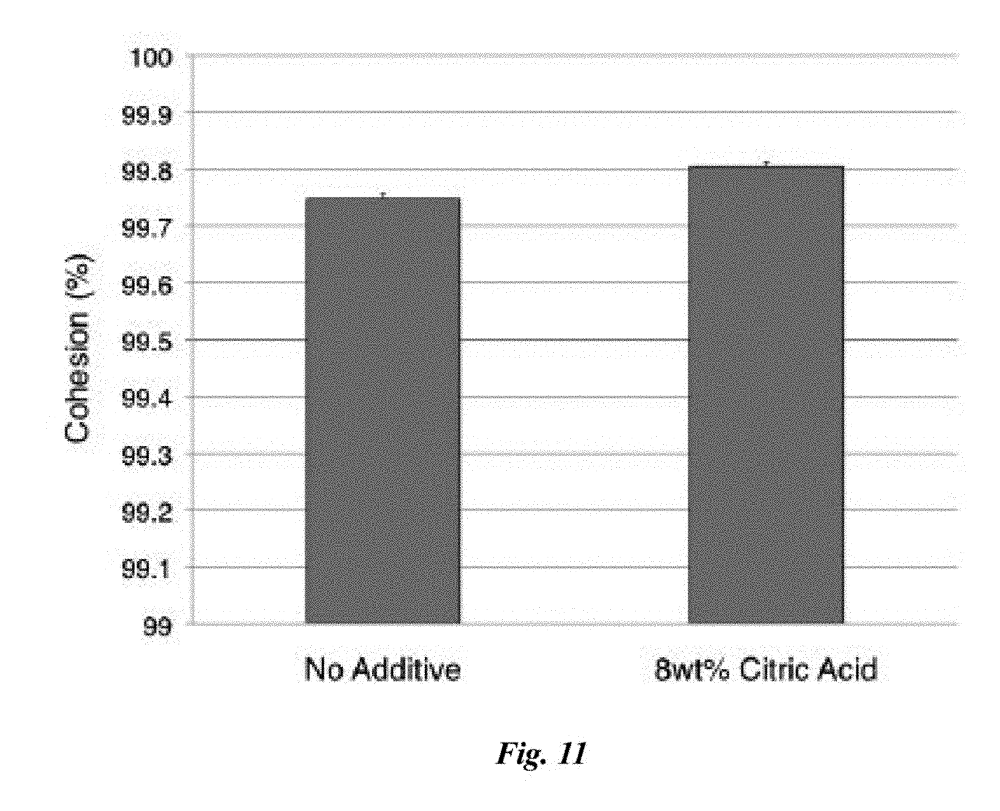

Examination of the cement cohesion (FIG. 11) revealed cohesion of 99.75% for cement made with no citric acid. The addition of 8 wt % CA resulted in a slight increase to 99.80%.

Testing of the injectability of the cements was performed for the various additions of CA to the powder.

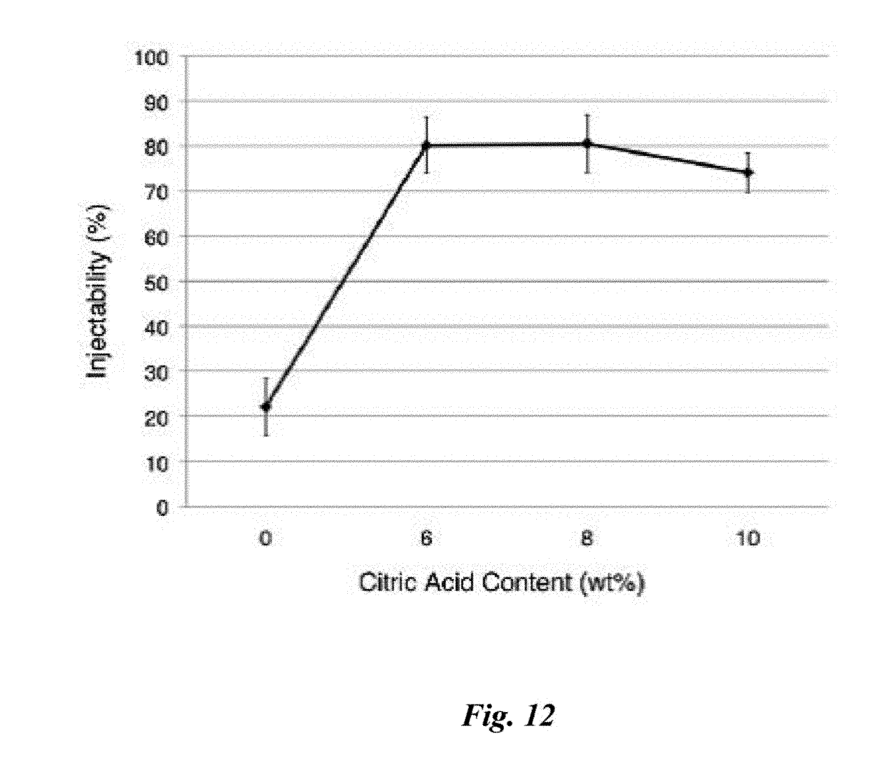

The powder with 0 wt % CA showed poor injectability, with an average injected amount of 22%. The addition of CA greatly increased the injectability of the cement to 80% for 6 wt % and 8 wt % CA and 74% for 10 wt % CA (FIG. 12). Qualitatively, the addition of citric acid to the cement powder resulted in cement pastes of much lower viscosity as compared to cement with no citric acid. The proportion ejected was in fact higher than 80% since residual cement remained in the nozzle of the syringe.

Discussion

Sodium Phosphate Solution Liquid Phase

The effect of the pH of the sodium phosphate solution on setting was similar to the effects found with the citric acid solutions used in the original cement system (see the section entitled “TRIMAGNESIUM PHOSPHATE CEMENT FOR BIOMEDICAL APPLICATIONS” above). Increases in pH increased the setting time of the cement though smaller differences in time were seen between different pH with sodium phosphates. The compressive strength and porosity also reflected the results found previously using the citric acid solution. The compressive strength with the use of sodium phosphates was slightly higher than that found using citric acid, however this may be attributed to the lower porosity of the sodium phosphate cements at the optimal solution pH. As found with the citric acid solutions, the pH of the sodium phosphate solution had little effect on the XRD profiles of the set cements and the setting reactions all resulted in a more amorphous material than the starting powder.

In contrast, the use of sodium phosphate was found to decrease the setting times of two hydroxyapatite-setting calcium phosphate cements, almost 50% for the traditional formulation, but subsequently decreased the injectability of the cement (Burguera 2006). Boudeville et al. had similar results in 1999 with an alternative formulation for a hydroxyapatite-forming cement using monocalcium phosphate monohydrate and calcium oxide.

The effect of their pH 7.4 sodium phosphate solution was pronounced, with decreases in setting time at a given powder-to-liquid ratio as the concentration of the solution increased. Examining only the case of water versus 1M sodium phosphate solution, the use of sodium phosphate resulted in an increase in compressive strength as well as a decrease in setting time. This represents the opposite effect found for our cement, with an increase in compressive strength accompanying an increase in setting time.

The effect of increasing solution pH was more noticeable in the change in crystal shape as seen in the SEM micrographs. As pH increased crystals became more plate-like, eventually forming blade-like structures at pH 7.0 and 8.8. The change in structure from larger more 3D crystal shapes to the more 2D plate structures was only slightly represented in the XRD patterns of the material with peaks becoming more pronounced with the pH 7.0 and 8.8 solutions. The mechanism for the change may be due to an increase in solubility of the starting material which upon dissolving in the solution re-precipitates as crystals with a preferred orientation. The increase in crystallinity may stem from the length of the setting time, with longer setting reactions allowing for the time-dependent act of forming regular crystals.

The Use of Citric Acid for Enhancement of Clinical Attributes

The addition of citric acid in powder form to the cement solid phase had a dramatic effect on the setting reaction. While 6 and 10 wt % citric acid resulting in the fastest setting times, it was the slight delay in reaching the final setting of the 8 wt % citric acid mixture that made it the optimal choice for a clinical setting. With an initial setting time of just under 5 minutes and final setting time around 16 minutes, the cement becomes cohesive enough to mix and place, while remaining in a quasi-plastic state for an additional 10 minutes, allowing the surgeon to fine-tune the fit of the material.

The compressive strength of the material decreased slightly with the addition of citric acid, except in the case of 6 wt %. The compressive strength of the 8 wt % formulation is on par with the results found in the section entitled “TRIMAGNESIUM PHOSPHATE CEMENT FOR BIOMEDICAL APPLICATIONS” above for the cements using a citric acid solution and is still within the desired strength range for bone cements. The 8 wt % cement showed an interesting relationship in terms of porosity, matching the result found for the cement without citric acid.

The phase analysis of the cements showed an increase in the presence of Newberyite (MgHPO₄.3H₂O) compared to the previous phases. It was shown that newberyite forms preferentially between pH 2.49 and 7.92, with bobierrite (Mg₃(PO₄)2.8H₂O) and magnesium orthophosphate (Mg(H₂PO₄)2.4H₂O) at higher and lower pH, respectively (Duff 1971). The generation of more pronounced newberyite peaks in the cement phases would seem to indicate that the local drop in pH at the site of dissolving citric acid particles in the solid phase would serve as the perfect sites for the formation of newberyite crystals.

The results of the more rapid setting reaction were seen in the SEM micrographs, with a marked reversion to the structure found with sodium phosphate solutions with pH below 7.0. The citric acid cements again showed crystallites stuck in an amorphous matrix, though crystallite size decreased with increasing citric acid percentage. The amorphous material around the crystallites does not have the smooth appearance seen without citric acid, but looks like small, connected and poorly formed blade structures. This may indicate competition between phases during setting, with the drop in pH caused by the citric acid particles preventing full formation of the blade-like crystals seen before.

The effect of citric acid addition on the cement handling was mixed. Though the sodium phosphate only cements showed slow setting times, they exhibited very high cohesion, with virtually no weight loss upon immersion. The addition of the citric acid therefore provided little gain in this property, but showed no detrimental effect on the cements ability to remain as immersed. The effect of addition was far more apparent in injectability testing. Cement injectability increased 4-fold from 22% to 80% with the addition of 6 and 8 wt % CA to the powder. The increase in injectability appeared to be from the lower viscosity of the cements with citric acid, which allowed them to be loaded and push from a syringe with ease.

Biocompatibility of the Cement Material

In Vivo Results

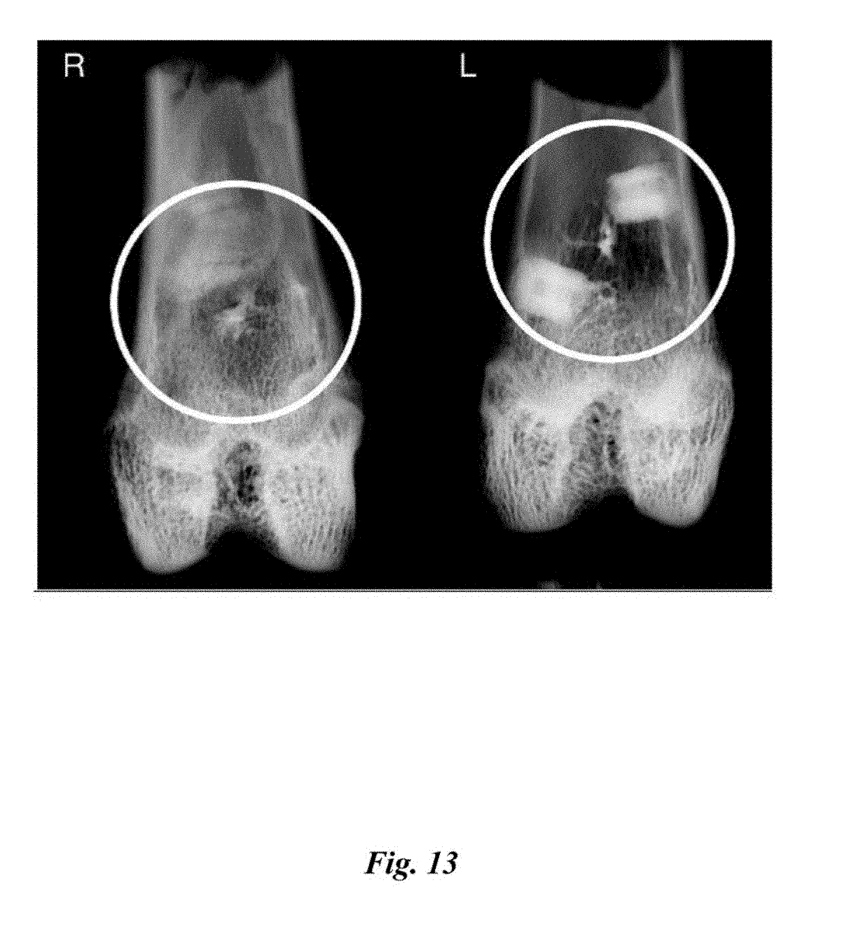

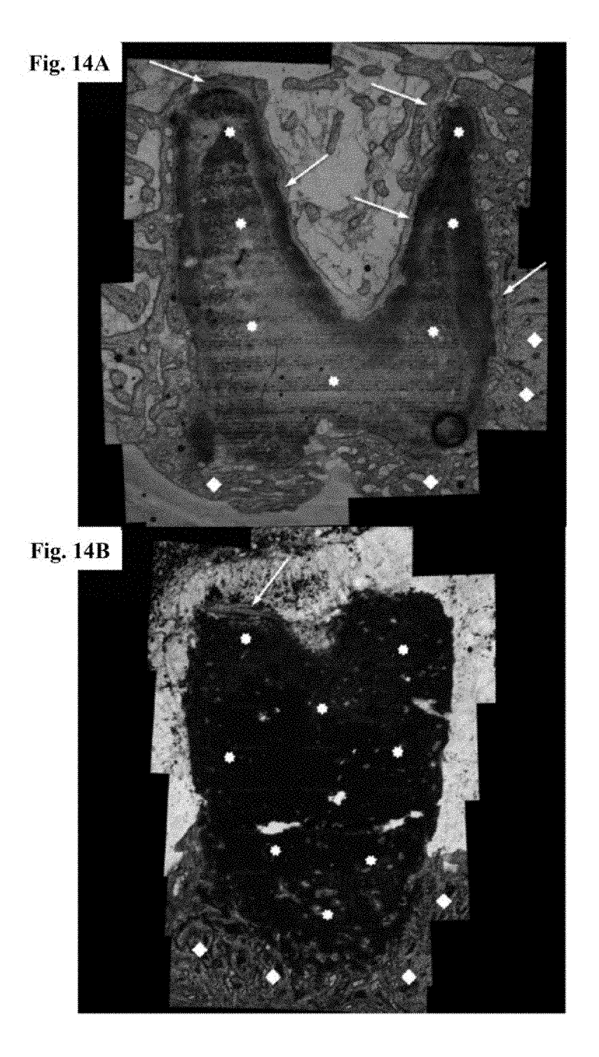

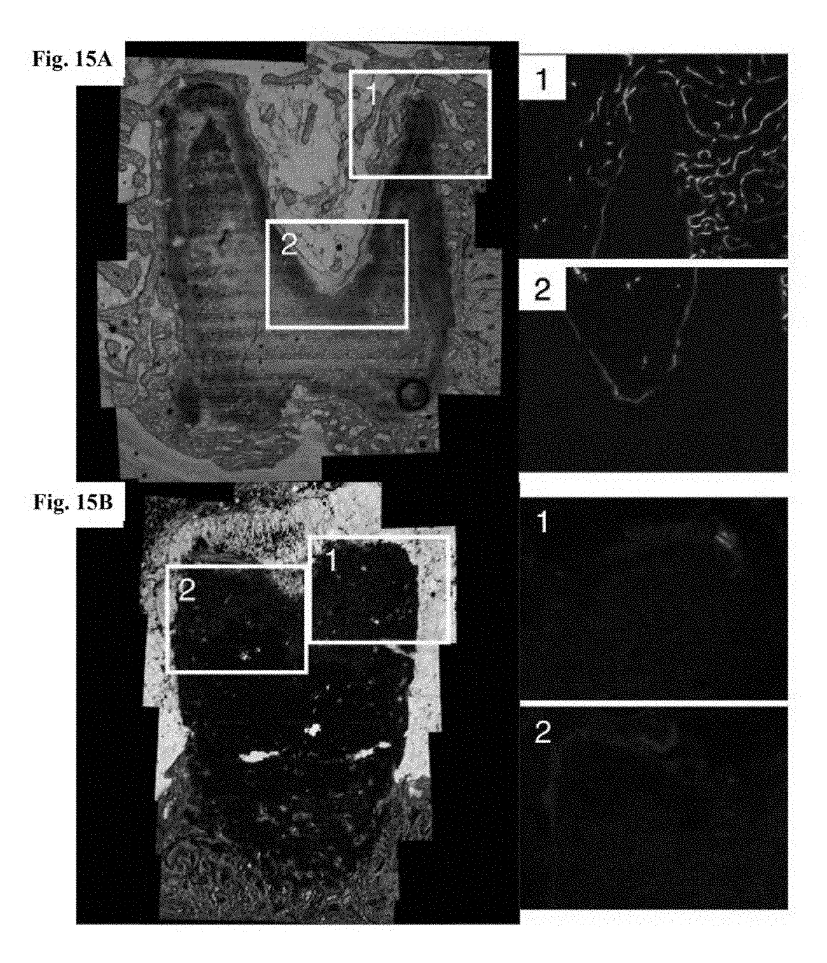

Preliminary in vivo studies have yielded promising results for the use of the above cement for bone repair. Three New Zealand White rabbits were given femoral defects which we filled with a preset cup shaped hollow magnesium phosphate cement of the invention (the above powder heat-treated at 600° C. mixed with 8 wt % citric acid powder together with a 1.0M solution of sodium phosphate monobasic and sodium phosphate dibasic at pH 7 at a powder-to-liquid ratio (P:L) of 1.0 g/ml) or with a preset brushite cement of the same shape and left to heal over 4 week's time. Histological analysis of the rabbit femurs revealed greater amounts of material resorption and bone growth around the remaining material for the magnesium phosphate. Brushite showed little resorption after 4 weeks time and no bone growth within the marker hole. (See FIGS. 13, 14 and 15A-15B)

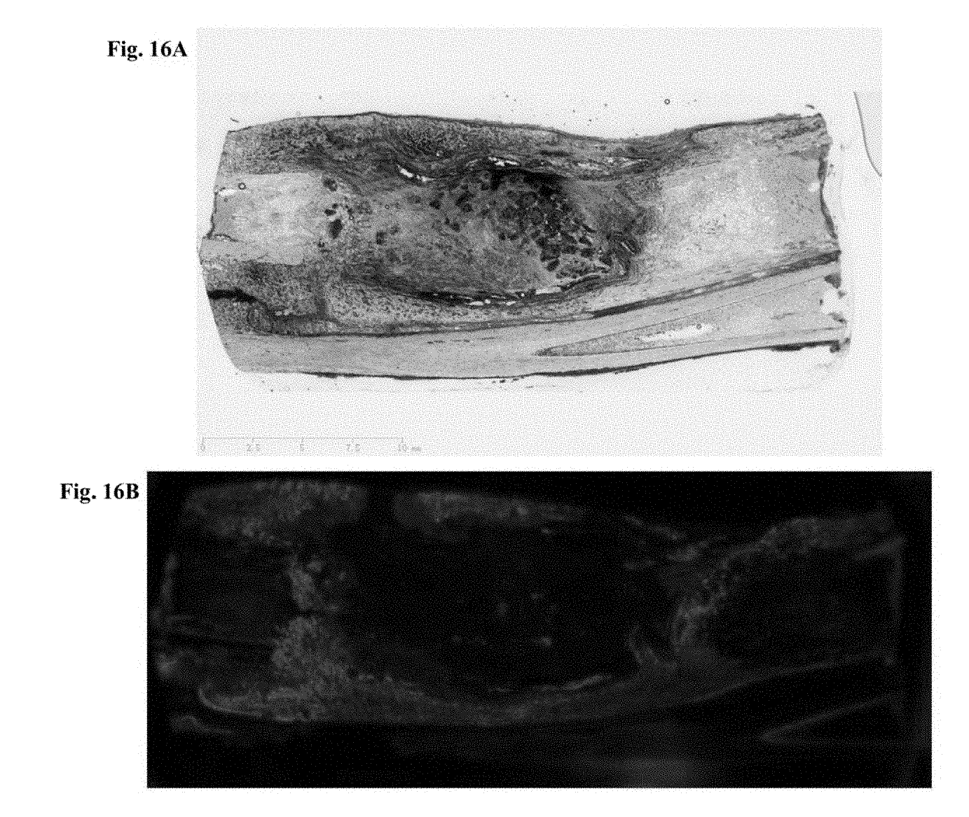

Also, 20 mm long bone defects were created in rabbit ulnae and filed with 0.5 mL of 300-1 mm granules made from cement powder synthesized as previously reported in the section entitled “TRIMAGNESIUM PHOSPHATE CEMENT FOR BIOMEDICAL APPLICATIONS” above. Only powder heat-treated at 600° C. was used and it was mixed with 8 wt % citric acid powder and the liquid cement reactant was a 1.0M solution of sodium phosphate monobasic and sodium phosphate dibasic at pH 7 at a powder-to-liquid ratio (P:L) of 1.0 g/ml. Immediately after mixing, the cement paste was cast and allowed to set prior to grinding and sieving. Fluorescent stain Alizarin Red (30 mg/kg) was injected at week 2, to show the temporal pattern of bone formation. FIG. 16A shows a light micrograph showing near complete repair of the cortical shaft of a 20 mm ulna defect after 4 weeks implantation, some granules of unresorbed cement are visible in the medullary cavity. FIG. 16B shows the pattern of extensive bone formation at weeks 2.

### Other Routes to Amorphous Magnesium Phosphates

### EXAMPLE 1

A Magnesium chloride solution (500 ml; 0.67 mM) was mixed with a solution of disodium phosphate (500 ml; 1.0 mM) at room temperature. The pH of the mixture was set at pH 10 by adding small aliquots of dilute solutions of sodium hydroxide and/or phosphoric acid. An amorphous precipitate formed in the solution within 24 hours. This precipitate was dehydrated at 200° C. for 30 minutes and formed a partially dehydrated trimagnesium phosphate that may contained traces of sodium. The partially dehydrated trimagnesium phosphate powder was mixed with distilled water in a powder to liquid ratio of 1:1 and the mixing paste set within 10 minutes to form a hard material.

### EXAMPLE 2

A Magnesium chloride solution (500 ml; 6.7 mM) was mixed with a disodium phosphate (500 ml; 10.0 mM) at 4° C. The pH of the mixture was set at pH 8 by adding small aliquots of dilute solutions of sodium hydroxide or phosphoric acid. An amorphous precipitate formed in the solution. This precipitate was heated at 220° C. for 1 hour and formed a partially dehydrated amorphous trimagnesium phosphate with traces of sodium. The partially dehydrated trimagnesium phosphate powder was mixed with a solution of citric acid (0.5M) in a powder to liquid ratio of 1:1, and the mixing paste set within 10 minutes to form a hard material.

### EXAMPLE 3

A magnesium chloride solution (0.67 mM) was mixed with a solution of disodium phosphate (1.0 mM) at room temperature. The pH of the mixture was set at pH 10 by adding small aliquots of dilute solutions of sodium hydroxide or phosphoric acid. A crystalline precipitate formed in the solution. This precipitate was mainly composed of the mineral cattiite, which is a trimagnesium phosphate hydrated with 22 molecules of water. The cattiite powder was heated at 220 C and formed a partially dehydrated amorphous powder. The amorphous powder contained traces of sodium. The amorphous powder was mixed with a phosphate buffer solution (0.1M) in a powder to liquid ratio of 1:1. The mixture resulted in a paste that set to form a solid ceramic material within 7 minutes.

### Advanced Applications for Magnesium Phosphate Cement

The inventors investigated the above magnesium phosphate cement formulation as a coating. Three different rod etching techniques were used and the rods were dip coated in cement slurries at 3 different powder-to-liquid ratios (P:L). Dip coating of the rods with cement slurries of various powder-to-liquid ratios (P:Ls) resulted in coatings which increased in thickness with a corresponding increase in P:L. The degradation of the coating formulations showed controlled dissolution in PBS, completely dissolving in 80-110 days.

5.1.3 Materials and Methods

Materials

Trimagnesium phosphate pentahydrate (Mg₃(PO₄)2.5H₂O; TMPP) powder was obtained from Jost Chemical (St. Louis, Mo., USA), citric acid (CA), sodium nitrate, and sodium phosphate monobasic were obtained from Fisher Scientific (Ottawa, ON, Canada) and sodium phosphate dibasic was obtained from Sigma-Aldrich (Oakville, ON, Canada).

Methods

Titanium rods (50 mm×5 mm) were etched and functionalized according to procedures outlined in previous publications (Vetrone 2009, Jonasova 2008). In brief, rods were immersed in sulphuric acid or a 50:50 mixture of sulphuric acid and tert-butyl hydroperoxide for 24h with one rod left untreated. A treatment summary is found in Table 5.1. After etching, all three rods were soaked in 10M NaOH at 60° C. for 24 hours to functionalize the material surface. The etched rods were examined using scanning electron microscopy (SEM) and electron-dispersive X-rays (EDX) to visualize the surface morphology and chemistry.

TABLE 5.1
Treatments for the titanium rods

| Rod | Treatment |
|---|---|
| 1 | Untreated (No Etching) |
| 2 | Sulphuric Acid |
| 3 | Sulphuric Acid/Tert-butyl |
| | hydroperoxide |

The treated rods were cut into thirds using a diamond saw and dip-coated in a magnesium phosphate cement formulation, previously described in the above section entitled “INVESTIGATION INTO THE OPTIMIZATION OF THE MAGNESIUM PHOSPHATE CEMENT SYSTEM”, at powder-to-liquid ratios (P:L) of 0.25, 0.33 and 0.50 g/ml. The coated rods were resin embedded, sectioned and polished. SEM and EDX were used to visualize and characterize the coating thickness.

Dissolution of the coating formulations was examined using cylindrical samples (6 mm ø×12mm) made at the three P:L and immersed in 50 mL of de-ionized water or PBS changed daily until complete dissolution was reached. Samples were patted with a damp paper towel to remove any extraneous surface liquid and weighed before the media was exchanged.

Vacuum dried cement was crushed in a mortar and pestle and added in a small amount to 1 mM solutions of sodium nitrate at pH from 1.0-8.0 and the surface charge was determined through measurement of the zeta potential (Zetasizer Nano—ZS, Malvern Instruments Ltd, Worcestershire, UK).

Results

Rod Treatments

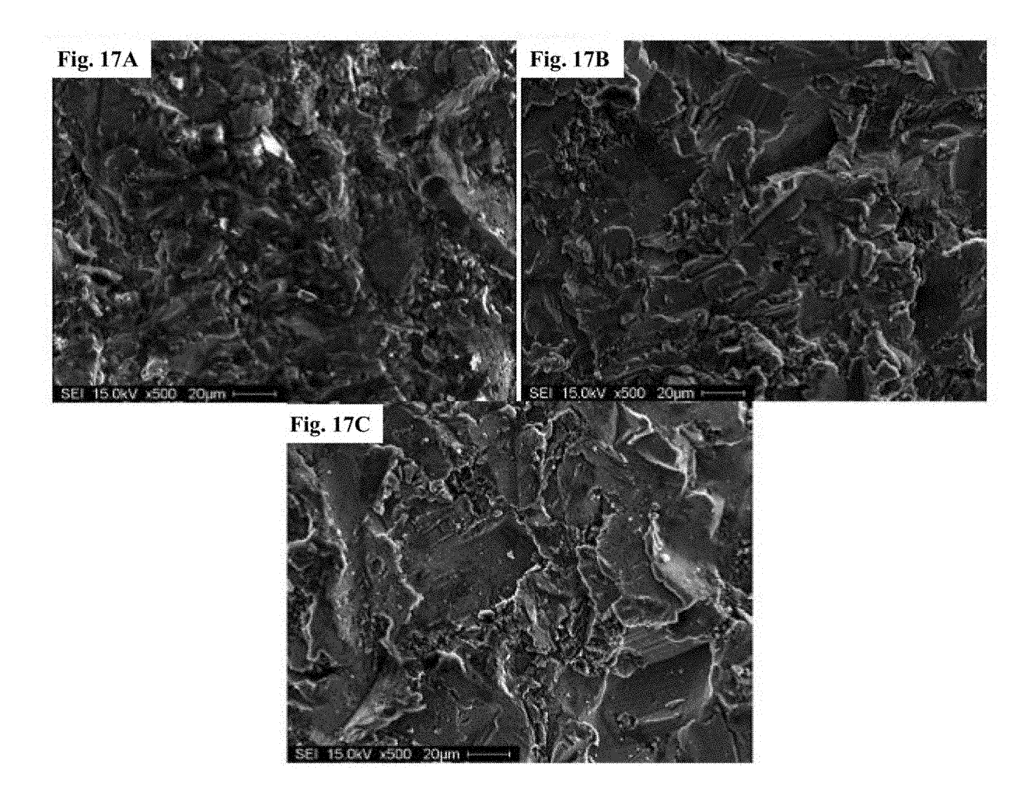

Etching of the rods showed little difference in surface morphology at low magnifications, as seen in FIGS. 17A-17C.

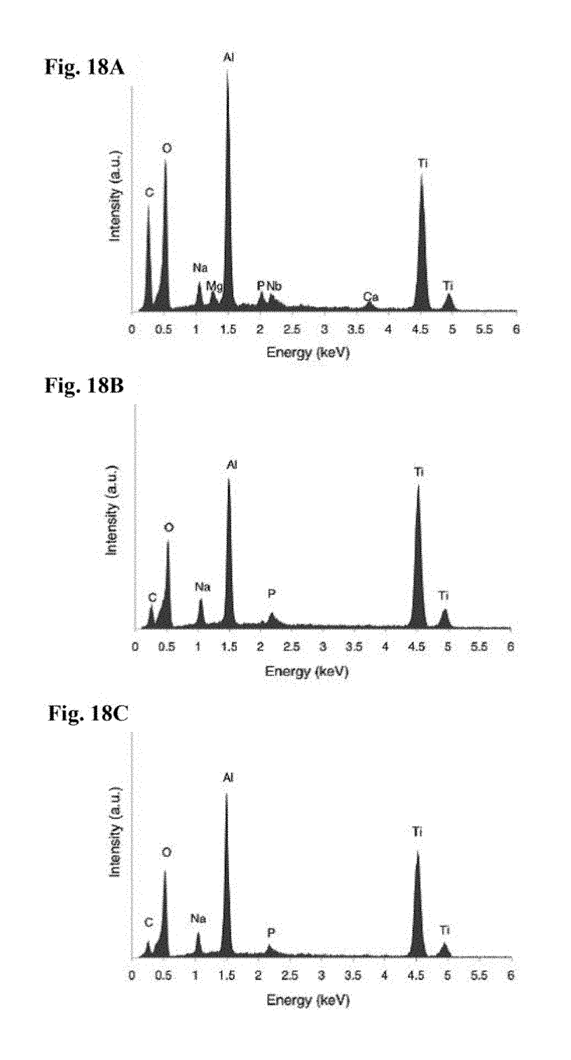

Examination of the surface chemistry of the etched rods using EDX revealed differences in surface chemistry between the different etching techniques. The untreated rod (FIG. 18A) showed significant oxygen and carbon peaks, a tall, well-defined aluminium peak and a slightly smaller titanium peak. The sulphuric acid treated rod (FIG. 18B) revealed less intense aluminium, carbon and oxygen peaks. The intensity of the titanium peak showed no change between the untreated and sulphuric rods. The sulphuric/peroxide rod (FIG. 18C) showed a similar decrease in the intensity of the aluminium peak as found with the sulphuric acid treatment. As well, the carbon and oxygen peaks were similar to those of the sulphuric treatment. The main difference between the two treatments was found in a small reduction in intensity of the titanium peak. Interestingly, the sodium peak for all three treatments remained at the same intensity.

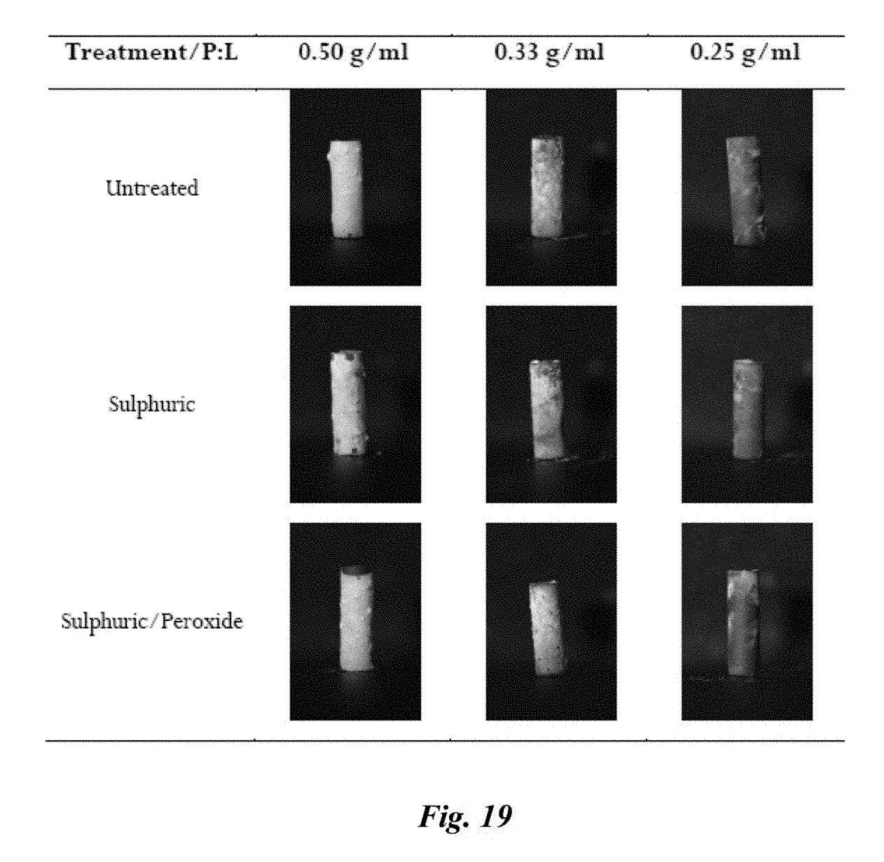

Dip coating of the rods yielded even coatings along the long axis of the rods. Small areas of thicker material were seen at the bottom of the rods where excess slurry had run during the curing stage. As expected, differences in coating thickness were seen with increasing P:L across all surface treatments, as seen in FIG. 19.

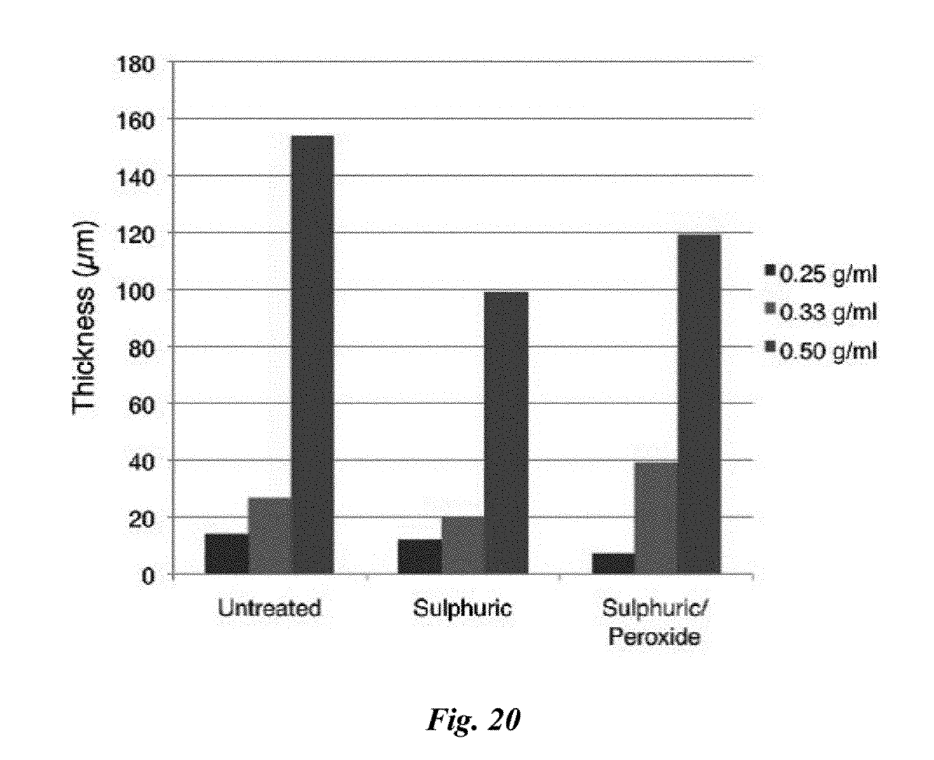

Examination of the coating upon resin embedding and sectioning, using EDX line scans confirmed the visual results seen across the P:L. The thickness of each coating remained relatively standard for each P:L, regardless of the surface treatment (FIG. 20). It is believed however that due to the nanotopography with the sulphuric/peroxide treatment, the coating would have the greatest mechanical adherence due to mechanical interlocking.

Figure Cement Dissolution

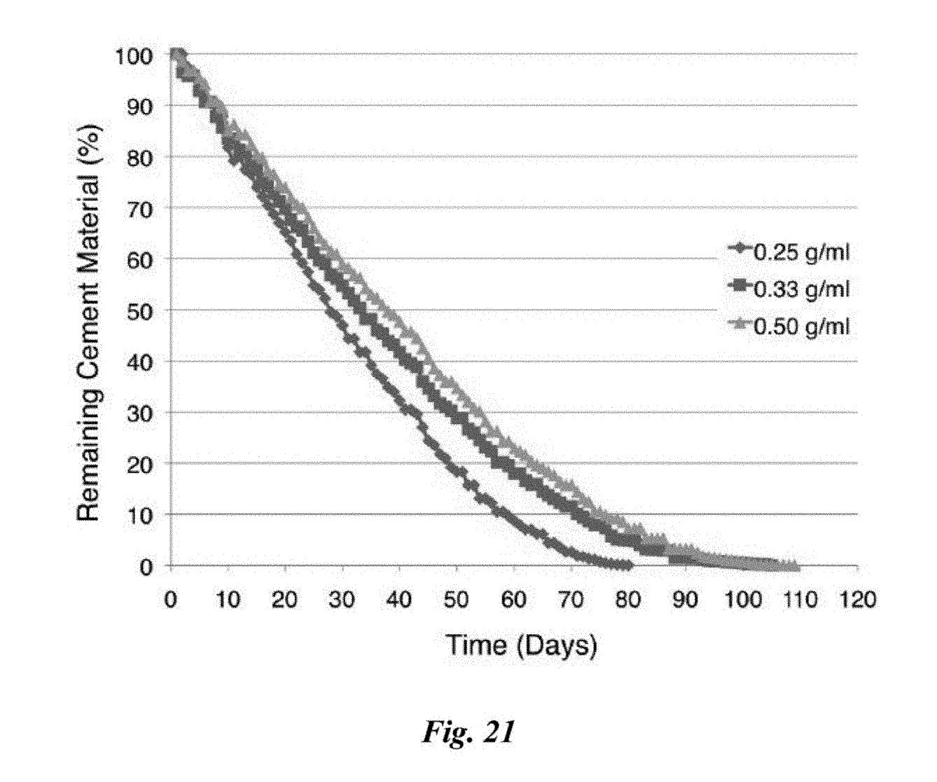

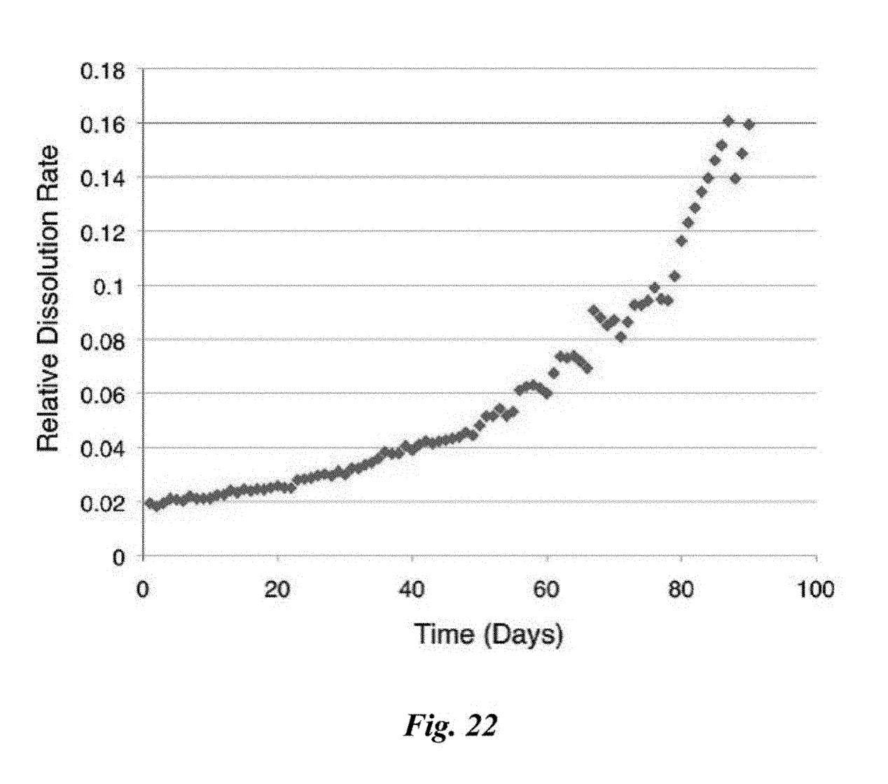

The cement showed a controlled dissolution rate at all P:L, with lower P:L showing higher dissolution rates due to greater porosity (FIG. 21). The rate of dissolution increased over time due the increasing ratio of medium to cement volume. The initial dissolution profiles of all P:L were linear over the first 50 days, before slowing with respect to the initial pellet mass. Examination of the percentage lost with respect to the previous mass (FIG. 22) showed an increase in the weight loss rate as the volume of medium to cement increased.

Discussion

Rod Etching and Coating

Etching of the rods showed little difference in topography at 500× magnification, and showed no evidence of the nanotopography from the surface treatments. The techniques, originally investigated for greater cell adhesion, were used in this study to try and create more attractive surfaces both for cells, material adhesion and the formation of bone. The EDX analysis of the surface also failed to reveal any large differences in the surface compositions. It does appear the two etching treatments done for the sulphuric and peroxide rods resulted in a reduction of impurities, such as magnesium, calcium and carbon, at the surface, possibly due to a reaction forming soluble compounds removed with the disposal of the etching solutions.

While the coating of orthopaedic implants with osteoconductive materials has been done for some time, many of the methods by which it is done are energy intensive, have many steps and require specialized equipment such as: plasma spraying, electrophoretic deposition, electrochemical deposition, sputter deposition and sol-gel deposition. Herein, a simple dip coating method allows to control of the coating thickness through the P:L of the cement slurry. The process of coating the implant takes only a few seconds, after which the cement material will set at room temperature, removing the need for high temperature calcining as found in other methods.

The thickness of the coating was controlled through changes in the cement P:L, resulting in changes in the viscosity of the slurry and cohesion of the coating. Interestingly though, the etching treatments for the rods did not show a clear trend in influencing the thickness of the coatings on the rods.

Cement Dissolution

The cements showed a controlled rate of dissolution around 100 days for all three P:L investigated. In comparison to in vitro degradation studies of brushite, our cement degraded at a much faster rate, losing 40-50% of its mass by 28 days, whereas brushite in a similar volume would have lost only 15% and have begun to plateau in PBS. Alternatively, the dissolution of brushite in serum showed results similar to those found using our cement and showed no sign of a plateau (Grover 2003). Grover also found similar results in 2006, examining the dissolution of a β-TCP/pyrophosphoric acid cement. The pyro-cement showed slow resorption in PBS, losing only 20% mass by 90 days time, however it did not show the plateau seen previously with brushite. In serum, the pyro-cement again showed slow dissolution, reaching just over 40% remaining mass by 90 days (Grover 2006). Brushite is known for its transformation into hydroxyapatite when immersed in an aqueous environment which slows its dissolution, resulting in the plateau (Grover 2003). Though no magnesium apatite exists, magnesium is well known to be a substitutional atom within hydroxyapatite. Due to the use of calcium and magnesium-free PBS, it is unknown whether an apatite product would form in a more complex ionic system.

### Magnesium Phosphate Granule Cements Preparation

Magnesium phosphate (tribasic pentahydrate, JOST, SS-13061) was placed in a crucible and heated to 625° C. for 30 minutes, then cooled down to room temperature for use. Citric acid anhydrous (Fisher, A940-500) was ground in a pestle and mortar into fine powder and sanitised by UV for 5 minutes. Cement mixing buffer was freshly made by adding acidic sodium phosphate monobasic (1 mol/l, Fisher BP 330-500) to alkaline sodium phosphate dibasic (1 mol/l, Fisher BP373-500), adjust to pH 7.0 and filtered through a 0.2 μm filter in a culture hood. Magnesium phosphate cements were prepared in a culture hood by mixing the previously prepared magnesium phosphate (1.84 g), citric acid (0.16 g) together with 2.5 ml of buffer. Solid to liquid ratio was 2 to 2.5. Magnesium phosphate cements were set about 5 minutes. Cements were dried in low vacuum pressure at 35° C. for 1.5 hours, and then in room temperature overnight. Dried cements were ground and granules (500-1000 μm) were obtained by sieving.

The animals (female New Zealand White rabbits) were prepared in a standard surgical fashion for a postero-lateral lumbar spine approach. Posterolateral intertransverse process fusions were performed. Approximately 3 cc of the granules were placed between the burred transverse processes and then closed in a routine surgical manner. At 4 weeks post-surgery a dose of 25 mg/Kg of tetracycline was administered. Following eight weeks of implantation, the animals were tranquilized and euthanized.

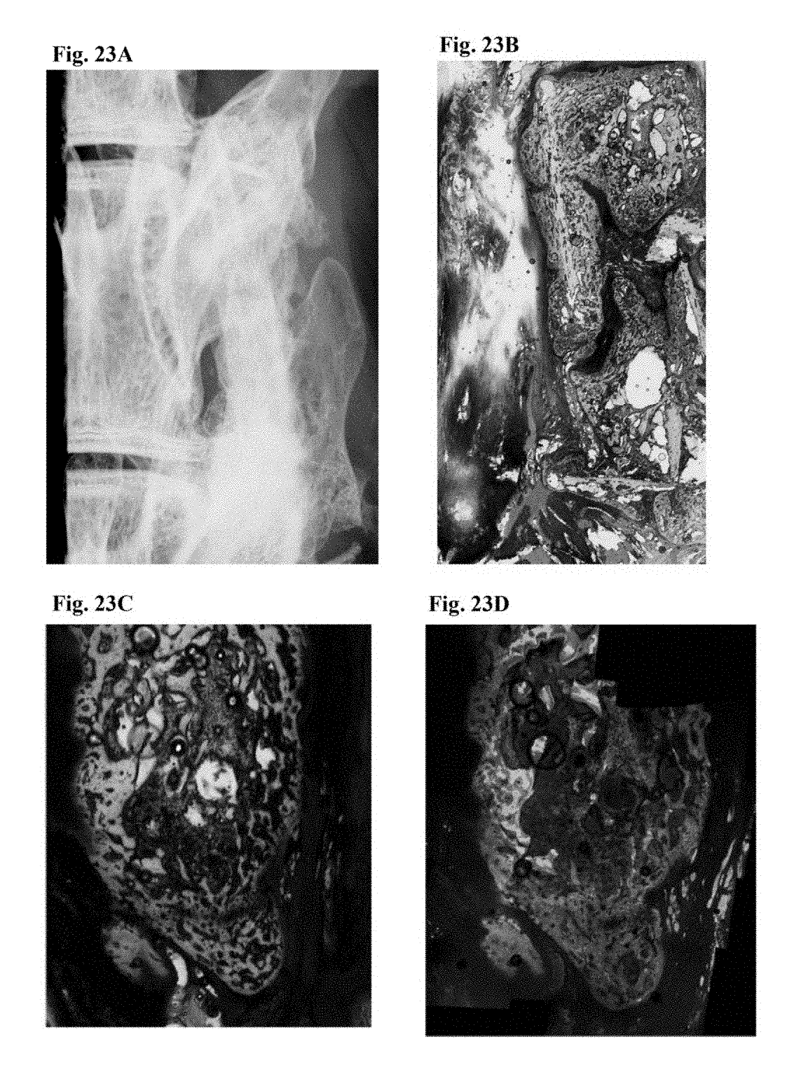

X-ray examination of explants showed dense material spanning the transverse processes (arrows) (FIG. 23A), and histological examination confirmed this to be bone tissue (arrows) (FIG. 23B) with isolated regions of material remaining. Higher magnification showed new bone with typical osteon features (FIG. 23C) formation of which at least partially occurred in the first month of implantation as confirmed by fluorescent markers stained bright green (FIG. 23D)(compare arrows in 23C and 23D).

The scope of the claims should not be limited by the preferred embodiments set forth in the examples, but should be given the broadest interpretation consistent with the description as a whole.

### REFERENCES

The present description refers to a number of documents, the content of which is herein incorporated by reference in their entirety.
Alkhraisat M H, Marino F T, Retama J R, Jerez L B, Lopez-Cabarcos E. Beta-tricalcium phosphate release from brushite cement surface. J Biomed Mater Res Part A. 2008; 84A(3): 710-7
Aramendia M A, Borau V, Jimenez C, Marinas J M, Romero F J. Synthesis and Characterization of Magnesium Phosphates and Their Catalytic Properties in the Conversion of 2-Hexanol. J Colloidal Interface Sci. 1999 September; 217(2): 288-98
Armstrong, S.; Pereverzev, A.; Dixon, S. J.; Sims, S. M. Activation of P2X7 receptors causes isoform-specific translocation of protein kinase C in osteoclasts. J. Cell. Sci. 2009; 122:136-144
Barralet J E, Grover L M, Gbureck U. Ionic modification of calcium phosphate cement viscosity. Part 2: hypodermic injection and strength improvement of brushite cement. Biomaterials. 2004; 25(11): 2197-203
Bohner M, Gbureck U, Barralet J E. Technological issues for the development of more efficient calcium phosphate bone cements: A critical assessment. Biomaterials. 2005 November; 26(33):6423-9
Brunner T J, Grass R N, Bohner M, Stark W J. Effect of particle size, crystal phase and crystallinity on the reactivity of tricalcium phosphate cements for bone reconstruction. J Mater Chem. 2007; 17(38):4072-8
Brunski J B. Chapter 2.9 Metals. In: Ratner B D, Hoffman A S, Schoen F J, Lemons J E, editors. Biomaterials Science: An Introduction to Materials in Medicine. Elsevier Ltd; 2004, p. 137
Burguera E F, Xu H H K, Weir M D. Injectable and Rapid Setting Calcium Phosphate Bone Cement with Dicalcium Phosphate Dihydrate. J Biomed Mater Res Part B. 2006; 77B(1): 126-134
De Long W G, Einhorn T A, Koval K, McKee M, Smith W, Sanders R, Watson T. Bone Grafts and Bone Graft Substitutes in Orthopaedic Trauma Surgery: A Critical Analysis. J. Bone Joint Surg. 2007; 89A(3): 649-58
Duff E J. Orthophosphates, Part VIII: The Transformation of Newberyite into Bobierrite in Aqueous Alkaline Solutions. J Chem Soc. (A). 1971: 2736-40
Eppley B L, Pietrzak W S, Blanton M W. Allograft and Alloplastic Bone Substitutes: A Review of Science and Technology for the Craniomaxillofacial Surgeon. J Craniofacial Surg. 2005; 16(6): 981-9
Gbureck U, Barralet J E, Radu L, Klinger H G, Thull R. Amorphous alpha-Tricalcium Phosphate: Preparation and Aqueous Setting Reaction. J Amer Ceram Soc; 2004 June; 87(6):1126-32
Grover L M, Knowles J C, Fleming G J P, Barralet J E. In vitro ageing of brushite calcium phosphate cement. Biomaterials. 2003; 24(23): 4133-41
Grover L M, Gbureck U, Wright A J, Tremayne M, Barralet J E. Biologically mediated resorption of brushite cement in vitro. Biomaterials. 2006; 27(10): 2178-85
Gulotta L V, Kovacevic D, Ying L, Ehteshami J R, Montgomery S, Rodeo S A. Augmentation of Tendon-to-Bone Healing With a Magnesium-Based Bone Adhesive. Am J Sport Med. 2008 July; 36(7):1290-7
Hallab N J, Jacobs J J, Katz J L. Chapter 7.7 Orthopedic Applications. In: Ratner B D, Hoffman A S, Schoen F J, Lemons J E, editors. Biomaterials Science: An Introduction to Materials in Medicine. Elsevier Ltd; 2004, p. 540-1
Jonasova L, Muller F A, Helebrant A, Strnad J, Greil P. Biomimetic apatite formation on chemically treated titanium. Biomaterials. 2008; 25:1187-94
Kingery W D. II, Cold Setting Properties. J Amer Ceram Soc. 1950; 33(8)242-7
Lally T. Bio-Adhesive Composition, Method for Adhering Objects to Bone. U.S. Pat. No. 6,533,821-B1. 2000 Jun. 22
Kretlow J D, Young S, Klouda L, Wong M, Mikos A G. Injectable Biomaterials for Regenerating Complex Craniofacial Tissues. Adv. Mater. 2009; 21: 3368-3393
Lally T. Bio-Adhesive Composition, Method for Adhering Objects to Bone. U.S. Pat. No. 6,533,821-B1. 2000 Jun. 22
Leroux L, Hatim Z, Frèche M, Lacout J L. Effects of Various adjuvents (Lactic Acid, Glycerol and Chitosan) on the Injectability of a Calcium Phosphate Cement. Bone. 1999; 25(2 Suppl): 31-4S
Lucas G L, Cooke F W, Friis E A. Chapter 6.2 Stress Shielding of Bones by Implants. A Primer of Biomechanics. Springer-Verlag; 1999, p. 79-88
Pfaffl M W. A new mathematical model for relative quantification in real-time RT-PCR. Nucleic Acids Res. 2001; 29(9): e45
Pina S, Olhero S M, Gheduzzi S, Miles A W, Ferreira J M F. Influence of setting liquid composition and liquid-to-powder ratio on properties of a Mg-substituted calcium phosphate cement. Acta Biomaterialia. 2009; 5(4): 1233-40
Maurus P B, Kaeding C C. Bioabsorbable Implant Material Review. Oper Tech Sports Med. 2004; 12(3): 158-60
Poitout D G. Biomechanics and Biomaterials in Orthopedics. Springer-Verlag London Limited; 2004, p. 86-92
Schendel S A, Peauroi J. Magnesium-based Bone Cement and Bone Void Filler: Preliminary Experimental Studies. J Craniofac Surg. 2009 March; 20(2):461-4
Staiger M P, Pietak A M, Huadmai J, Dias G. Magnesium and its alloys as biomaterials: a review. Biomaterials. 2006; 27: 1728-34
Vetrone F, Variola F, de Oliviera P T, Zalzal S F, Yi J H, Sam J. Nanoscale Oxidative Patterning of Metallic Surfaces to Modulate Cell Activity and Fate. Nano Lett. 2009 February; 9(2): 659-65
Wagh, A S, editor. Chemically Bonded Phosphate Ceramics: 21st Century Materials with Diverse Applications. Oxford: Elsevier Ltd; 2004, p. 152
Wu F, Su J, Wei J, Guo H, Liu C. Injectable bioactive calcium-magnesium phosphate cement for bone regeneration. Biomed Mater. Doi: 10.1088/1748-6041/3/4/044105
Wu F, Wei J, Guo H, Chen F, Hong H, Liu C. Self-setting bioactive calcium-magnesium phosphate cement with high strength and degradability for bone regeneration. Acta Biomater. 2008 November; 4(6): 1872-84

1-36. (canceled)

37. A method for spine fusion, comprising the steps of:
providing granules comprising magnesium phosphate;
placing said granules between two vertebrae; and
letting resorption of said granules and growth of bone around remaining granules until fusion of said two vertebrae.

38. The method of claim 37, wherein said method provides for formation of new bone having osteon-like features.

39. The method of claim 37, wherein said method provides for formation of new bone within one month of implementation of said granules.

40. The method of claim 37, wherein said method provides for fusion of the two vertebrae within eight weeks of implementation of said granules.

41. The method of claim 37, wherein said granules have a size of 300 μm to 1 mm.

42. The method of claim 37, wherein said granules have a size of 500 μm to 1000 μm.

43. The method of claim 37, wherein said granules are free of calcium.

44. The method of claim 37, wherein said granules comprise trimagnesium phosphate.

45. The method of claim 37, wherein said granules are obtained from granulation of a set magnesium phosphate cement.

46. The method of claim 45, wherein said set cement comprises trimagnesium phosphate reacted with an aqueous solution.

47. The method of claim 46, wherein said aqueous solution comprises at least one of organic acid ions, monovalent cations, and phosphate ions.

48. The method of claim 46, wherein said aqueous solution comprises at least one of citric acid, citrate, sodium citrate, and sodium phosphate.

49. The method of claim 45, wherein said set cement displays a compressive strength of at least 15 MPa.

50. The method of claim 45, wherein said set cement displays a compressive strength of at least 15 MPa.

51. The method of claim 45, wherein said set cement displays a wet compressive strength of at least 19.1 MPa.

52. The method of claim 45, wherein said set cement comprises XRD peaks characteristic Farringtonite (Mg₃(PO₄)₂).
